SEQUENCE LISTING (15 sequences)

SEQ ID NO: 1 - protein, 226 aa, Escherichia coli
MNPIVINRLQRKLGYTFNHQELLQQALTHRSASSKHNERLEFLGDSILSYVIANALYHRFPRVDEGDMSRMRATLVRGNTLAELAREFELGECLRLGPGELKSGGFRRESILADTVEALIGGVFLDSDIQTVEKLILNWYQTRLDEISPGDKQKDPKTRLQEYLQGRHLPLPTYLVVQVRGEAHDQEFTIHCQVSGLSEPVVGTGSSRRKAEQAAAEQALKKLELE

SEQ ID NO: 2 - DNA, 355 bp, Artificial Sequence
  Description of Artificial Sequence  Synthetic Primer
tacgatttaggtgacactatagaatacacggaattaatacgactcactatagggaattaccctcactaaagggaggaagctgcaattgggatgcaagctttccacatctggcacagagcgggaggtctctgagccactgggcctagatgatgccggaaacaagaagtcatcaaagggttctgccttcagctccacgttgctgatgctcttgactggctccaaggatggcttgggctcagggtcgttgagaaggggcagggtgaaggcctcctcagactctggggtggaagcctcaggcagacctccagtcaaatccagggaggccacagacatctcctctgggaagccaagaatt

SEQ ID NO: 3 - DNA, 40 bp, Artificial Sequence
  Description of Artificial Sequence  Synthetic Primer
aatttaatacgactcactataggaagcattgagcaaatcc

SEQ ID NO: 4 - DNA, 41 bp, Artificial Sequence
  Description of Artificial Sequence  Synthetic Primer
aatttaatacgactcactataggcttctggccaggggtctc

SEQ ID NO: 5 - DNA, 41 bp, Artificial Sequence
  Description of Artificial Sequence  Synthetic Primer
aatttaatacgactcactataggtaccagaagcggtgccgg

SEQ ID NO: 6 - DNA, 43 bp, Artificial Sequence
  Description of Artificial Sequence  Synthetic Primer
aatttaatacgactcactataggcaaacgactgtcctggccgt

SEQ ID NO: 7 - DNA, 29 bp, Artificial Sequence
  Description of Artificial Sequence  Synthetic Primer
ggtaatacgactcactatagggagacagg

SEQ ID NO: 8 - DNA, 29 bp, Artificial Sequence
  Description of Artificial Sequence  Synthetic Primer
aagtggatattgttgccatcacctgtctc

SEQ ID NO: 9 - DNA, 29 bp, Artificial Sequence
  Description of Artificial Sequence  Synthetic Primer
aatgatggcaacaatatccaccctgtctc

SEQ ID NO: 10 - DNA, 27 bp, Artificial Sequence
  Description of Artificial Sequence  Synthetic Primer
aaggtcatccatgacaactcctgtctc

SEQ ID NO: 11 - DNA, 29 bp, Artificial Sequence
  Description of Artificial Sequence  Synthetic Primer
aaaaagttgtcatggatgacccctgtctc

SEQ ID NO: 12 - DNA, 30 bp, Artificial Sequence
  Description of Artificial Sequence  Synthetic Primer
aagcttcactggcatggccttccctgtctc

SEQ ID NO: 13 - DNA, 29 bp, Artificial Sequence
  Description of Artificial Sequence  Synthetic Primer
aagaaggccatgccagtgagccctgtctc

SEQ ID NO: 14 - DNA, 29 bp, Artificial Sequence
  Description of Artificial Sequence  Synthetic Primer
aacagggtggtggacctcatgcctgtctc

SEQ ID NO: 15 - DNA, 29 bp, Artificial Sequence
  Description of Artificial Sequence  Synthetic Primer
aacatgaggtccaccaccctgcctgtctc

(19) United States
(12) Patent Application Publication
(10) Pub. No.: US 2012/0122217 A1
(43) Pub. Date: May 17, 2012

(54) METHODS AND COMPOSITIONS FOR REDUCING TARGET GENE EXPRESSION USING COCKTAILS OF siRNAS OR CONSTRUCTS EXPRESSING siRNAS

(75) Inventors: David Brown, Austin, TX (US); Lance P. Ford, Austin, TX (US); Rich Jarvis, Austin, TX (US)

(73) Assignee: APPLIED BIOSYSTEMS, LLC, CARLSBAD, CA (US)

(21) Appl. No.: 13/360,148

(22) Filed: Jan. 27, 2012

Related U.S. Application Data
(63) Continuation of application No. 13/039,653, filed on Mar. 3, 2011.
Application No. 13/039,653 is a division of application No. 10/298,480, filed on Nov. 15, 2002.
(60) Provisional application No. 60/402,347, filed on Aug. 10, 2002.

(51) Int. Cl. C12N 5/02

(52) U.S. Cl. 435/375

(57) ABSTRACT

The present invention concerns methods and compositions involving the production or generation of siRNA mixtures or pools capable of triggering RNA-mediated interference (RNAi) in a cell. Compositions of the invention include kits that include reagents for producing or generating siRNA pools. The present invention further concerns methods using polypeptides with RNase III activity for generating siRNA mixtures or pools that effect RNAi, including the generation of a number of RNA molecules to the same target gene.

This application claims the priority of U.S. Provisional Application Ser. No. 60/402,347, filed Aug. 10, 2002, the disclosures of which is specifically incorporated herein by reference in its entirety.

### BACKGROUND OF THE INVENTION

1. Field of the Invention

The present invention relates generally to the field of molecular biology. More particularly, it concerns methods and compositions for reducing or eliminating the expression of at least one target gene by obtaining and introducing into a cell multiple single or double stranded RNAs (dsRNAs) or DNA constructs capable of expressing multiple siRNAs in cells. The collections of multiple siRNAs or DNA constructs capable of expressing multiple siRNAs are referred to as cocktails or pools. The cocktails will typically be capable of reducing target gene expression in vitro or in vivo.

2. Description of the Related Art

RNA interference (RNAi), originally discovered in Caenorhabditis elegans by Fire and Mello (Fire et al., 1998), is a phenomenon in which double stranded RNA (dsRNA) reduces the expression of the gene to which the dsRNA corresponds. The phenomenon of RNAi was subsequently proven to exist in many organisms and to be a naturally occurring cellular process. The RNAi pathway can be used by the organism to inhibit viral infections, transposon jumping and to regulate the expression of endogenous genes (Huntvagner et al., 2001; Tuschl, 2001; Waterhouse et al., 2001; Zamore 2001). In original studies, researchers were inducing RNAi in non-mammalian systems and were using long double stranded RNAs. However, most mammalian cells have a potent antiviral response causing global changes in gene expression patterns in response to long dsRNA, thus arousing questions as to the existence of RNAi in humans. As more information about the mechanistic aspects of RNAi was gathered, RNAi in mammalian cells was shown also to exist.

Using several different systems, it was observed that long dsRNAs are processed into shorter small interfering RNA (siRNA) by a cellular ribonuclease containing RNaseIII motifs (Bernstein et al., 2001; Grishok et al., 2001; Hamilton and Baulcombe, 1999; Knight and Bass, 2001; Zamore et al., 2000). Genetics studies done in C. elegans, N. crassa and A. thaliana have lead to the identification of additional components of the RNAi pathway. These genes include putative nucleases (Ketting et al., 1999), RNA-dependent RNA polymerases (Cogoni and Macino, 1999a; Dalmay et al., 2000; Mourrain et al., 2000; Smardon et al., 2000) and helicases (Cogoni and Macino, 1999b; Dalmay et al., 2001; Wu-Scharf et al., 2000). Several of these genes found in these functional screens are involved not only in RNAi but also in nonsense mediated mRNA decay, protection against transposon-transposition (Zamore, 2001), viral infection (Waterhouse et al., 2001), and embryonic development (Hutvagner et al., 2001; Knight and Bass, 2001). In general, it is thought that once the siRNAs are generated from longer dsRNAs in the cell by the RNaseIII like enzyme, the siRNAs associate with a protein complex. The protein complex also called RNA-induced silencing complex (RISC), then guides the smaller 21 base double stranded siRNA to the mRNA where the two strands of the double stranded RNA separate, the antisense strand associates with the mRNA and a nuclease cleaves the mRNA at the site where the antisense strand of the siRNA binds (Hammond et al., 2001). The mRNA is subsequently degraded by cellular nucleases.

Elbashir et al. (2001) discovered that siRNAs are sufficient to induce gene specific silencing in mammalian cells. In one set of experiments, siRNAs complementary to the luciferase gene were co-transfected with a luciferase reporter plasmid into NIH3T3, COS-7, HeLaS3, and 293 cells. In all cases, the siRNAs were able to specifically reduce luciferase gene expression. In addition, the authors demonstrated that siRNAs could reduce the expression of several endogenous genes in human cells. The endogenous targets were lamin A/C, lamin B1, nuclear mitotic apparatus protein, and vimentin. The use of siRNAs to modulate gene expression has now been reproduced by at least two other labs (Caplen et al., 2001; Hutvagner et al., 2001) and has been shown to exist in more that 10 different organisms spanning a large spectrum of the evolutionary tree. RNAi in mammalian cells has the ability to rapidly expand our knowledge of gene function and cure and diagnose human diseases. However, much about the process is still unknown and thus, additional research and understanding will be required to take full advantage of it.

The making of siRNAs has been through direct chemical synthesis, through processing of longer double stranded RNAs by exposure to Drosophila embryo lysates, through an in vitro system derived from S2 cells, using phage RNA polymerase, RNA-dependant RNA polymerase, and DNA based vectors. Use of cell lysates or in vitro processing may further involve the subsequent isolation of the short, 21-23 nucleotide siRNAs from the lysate, etc., making the process somewhat cumbersome and expensive. Chemical synthesis proceeds by making two single stranded RNA-oligomers followed by the annealing of the two single stranded oligomers into a double stranded RNA.

WO 99/32619 and WO 01/68836 suggest that RNA for use in siRNA may be chemically or enzymatically synthesized. The enzymatic synthesis contemplated is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25 mer lengths with the endogenous nuclease complex that converts long dsRNAs to siRNAs in vivo. They do not describe or present data for synthesizing and using in vitro transcribed 21-25 mer dsRNAs. No distinction is made between the expected properties of chemical or enzymatically synthesized dsRNA in its use in RNA interference.

Similarly, WO 00/44914 suggests that single strands of RNA can be produced enzymatically or by partial/total organic synthesis. Preferably, single stranded RNA is enzymatically synthesized from the PCR products of a DNA template, preferably a cloned cDNA template and the RNA product is a complete transcript of the cDNA, which may comprise hundreds of nucleotides. WO 01/36646 places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences. U.S. Pat. No. 5,795,715 was filed Jun. 17, 1994, well before the phenomenon of RNA interference was described by Fire, et al. (1998). The production of siRNA was therefore, not contemplated by these authors.

In the provisional patent application 60/353,332, which is specifically incorporated by reference, the production of siRNA using the RNA dependent RNA polymerase, phage polymerase P2 (P2) and that this dsRNA can be used to induce gene silencing. Although this method is not commercially available or published in a scientific journal it was determined to be feasible. Several laboratories have demonstrated that DNA expression vectors containing mammalian RNA polymerase III promoters can drive the expression of siRNA that can induce gene-silencing (Brummelkamp et al., 2002; Sui et al., 2002; Lee et al., 2002; Yu et al., 2002; Miyagishi et al., 2002; Paul et al., 2002). The RNA produced from the RNA polymerase III promoter can be designed such that it forms a predicted hairpin with a 19-base stem and a 3-8 base loop. The approximately 45 base long siRNA expressed as a single transcription unit folds back on it self to form the hairpin structure as described above. Hairpin RNA can enter the RNAi pathway and induce gene silencing. The siRNA mammalian expression vectors have also been used to express the sense and antisense strands of the siRNA under separate polymerase III promoters. In this case, the sense and antisense strands must hybridize in the cell following their transcription (Lee et al., 2002; Miyagishi et al., 2002). The siRNA produced from the mammalian expression vectors whether a hairpin or as separate sense and antisense strands were able to induce RNAi without inducing the antiviral response. More recent work described the use of the mammalian expression vectors to express siRNA that inhibit viral infection (Jacque et al., 2002; Lee et al., 2002; Novina et al., 2002). A single point mutation in the siRNA with respect to the target prevents the inhibition of viral infection that is observed with the wild type siRNA. This suggests that siRNA mammalian expression vectors and siRNA could be used to treat viral diseases.

A typical project incorporating siRNA begins with the identification of an mRNA target site that is susceptible to siRNA-induced degradation. Approximately, half of the siRNAs designed to a particular target provide a 50% or greater reduction in gene expression. Approximately 25% provide 75% or greater reduction in gene expression. Screening for siRNAs will almost always lead to the identification of an effective siRNA, but the screening process is slow and labor intensive. A siRNA synthesis method that would get around transfecting 4 or more separate siRNA per target would be beneficial in cost and time. Thus, a method for attaining a greater reduction in gene expression is needed.

As described above, only about half of the candidate siRNAs, which may designate a dsRNA that may or may not effect RNAi to some degree, designed to a particular target provide a 50% or greater reduction in gene expression and approximately 25% provide 75% or greater reduction in gene expression. Not all dsRNA or candidate siRNA molecules can effect RNA interference of a target gene. The variation of efficacy in dsRNA in reducing or eliminating target gene expression may be attributed to the character of the dsRNA sequence and it target site and/or may be affected by accessibility of the target sequence. To date the design of an effective siRNA is determined empirically, which requires time and labor for screening and verification of RNAi activity. It would be advantageous to increase the frequency with which siRNAs reduce the expression of target genes. There are a number of studies that have been undertaken to generate design rules for siRNAs, but there have been no publications to suggest that a set of rules is forthcoming. Furthermore, it is anticipated that a single set of rules will not be developed given the uncertainty of mRNA tertiary structure and protein binding sites in mammalian cells. Methods that improve the frequency with which target gene expression is reduced would reduce or even eliminate the need to validate that a candidate siRNA, siRNA or siRNA expressing construct is functional. The savings in time and expense to researchers would be enormous.

### SUMMARY OF THE INVENTION

The present invention includes methods and compositions for introducing multiple siRNAs targeting different regions of a gene that typically can greatly improve the likelihood that the expression of the target gene will be reduced. The inventors have found that the different candidate siRNAs or siRNAs do not interfere with the activities of others in the mixture and that in fact, there appears to be some synergy between the siRNAs. This is applicable not only to siRNAs but to DNA constructs designed to express siRNAs (Brummelkamp 2002). Certain embodiments of the invention alleviate the need to screen or optimize candidate siRNAs. To determine the functionality of a Candidate siRNA it must be screened, verified, and/or optimized. The screening, selection and/or optimization process of a specific siRNA is labor intensive and time consuming. Thus, various embodiments of the invention, as described herein, provide improved methods for the application of cocktails or pools of siRNA or candidate siRNAs in reducing or eliminating the expression of a target gene(s) by eliminating the need to identify any specific siRNA molecule(s) with a particular effectiveness, as well as providing methods that may increase the effectiveness of RNA interference. As used herein, a “candidate siRNA” is an siRNA that has not been tested for its functionality as an siRNA. It is also contemplated that siRNAs may be single or double stranded RNA molecules.

SiRNAs are small single or dsRNAs that do not significantly induce the antiviral response common among vertebrate cells but that do induce target mRNA degradation via the RNAi pathway. The term siRNA refers to RNA molecules that have either at least one double stranded region or at least one single stranded region and possess the ability to effect RNAi. It is specifically contemplated that siRNA may refer to RNA molecules that have at least one double stranded region and possess the ability to effect RNAi. Mixtures or pools of dsRNAs (siRNAs) may be generated by various methods including chemical synthesis, enzymatic synthesis of multiple templates, digestion of long dsRNAs by a nuclease with RNAse III domains, and the like. A “pool” or “cocktail” refers to a composition that contains at least two siRNA molecules that have different selectivity with respect to each other, but are directed to the same target gene. Two or more siRNA molecules that have different selectivity with respect to each other, but are directed to the same or different target gene(s) are defined as different siRNAs. Different siRNAs may overlap in sequence, contain two sequences that are contiguous or non-contiguous in the target gene. In some embodiments, a pool contains at least or at most 3, 4, 5, 6, 7, 8, 9, 10 or more siRNA molecules. A pool may include a mixture of dsRNAs, candidate siRNAs or siRNAs directed to 2, 3, 4, 5, 6, 7, 8, 9, 10 or more regions of a target transcript (single target pool) or it may be directed to 2, 3, 4, 5, 6, 7, 8, 9, 10 or more regions of 1, 2, 3, 4, 5, 6, 7, 8, 9, 10 or more target transcripts (multiple target pool). An “siRNA directed to” a particular region or target gene means that a particular siRNA includes sequences that results in the reduction or elimination of expression of the target gene, i.e., the siRNA is targeted to the region or gene. The pool in some embodiments includes one or more control siRNA molecules. In other embodiments a control siRNA molecule is not included in the pool. A pool of siRNA molecules may also contain various candidate siRNA molecules that do not reduce or eliminate expression of a target gene.

The term dsRNA, candidate siRNA, or siRNA pool or cocktail encompasses both single and multiple target pools. A region of a target gene is a contiguous or non-contiguous nucleotide sequence of a target gene, which may or may not overlap other target sequences on the target transcript. A pool of dsRNA or siRNA may contain various dsRNA that are capable of reducing or eliminating the expression of at least one target gene in a cell with various degrees of efficacy. The efficacy of a pool of dsRNA or siRNAs will typically be greater than the efficacy of any individual member of the pool. Also, the percentage of dsRNA or siRNA pools able to reduce or eliminate target gene expression is typically higher than that seen with a number of individual dsRNAs or siRNAs.

The inventors have observed that the presence of multiple dsRNAs, each of which reduce the expression of a target gene to some degree, as well as the presence of some dsRNAs, which do not effect target gene expression, may be administered as a pool without interference between members of the pool and typically results in an additive or synergistic reduction in target gene expression. Thus, the present invention is directed to compositions and methods involving generation and utilization of pools or mixtures of small, double-stranded RNA molecules that effect, trigger, or induce RNAi more effectively. RNAi is mediated by an RNA-induced silencing complex (RISC), which associates (specifically binds one or more RISC components) with dsRNA pools of the invention and guides the dsRNA to its target mRNA through base-pairing interactions. Once the dsRNA is base-paired with its mRNA target, nucleases cleave the mRNA.

In certain embodiments of the invention, multiple dsRNAs or siRNAs can be introduced into a cell to activate the RNAi pathway. In other embodiments, various individual dsRNAs with different sequences may be co-transfected simultaneously to effectively produce a pool or mixture of dsRNAs within a transfected cell(s). The effects of multiple siRNAs, as described herein are typically additive and may be synergistic in some cases. The effectiveness of a dsRNA pool is in contrast to the information published in the literature that co-transfecting an active and an inactive siRNA reduced the effectiveness of the active siRNA ((Holen et al. 2002 Co-transfecting multiple siRNAs may greatly improve the effectiveness of reducing target gene expression and minimizes or eliminates the need to confirm siRNA activity of one or more dsRNA prior to use. The inventors have found that co-transfecting at least 4 siRNAs per target will reduce gene expression by at least 50% greater than 95% of the time. The dsRNAs and/or siRNAs can be prepared and introduced into cells in any way known to a person of ordinary skill in the art. In some embodiments, siRNA or dsRNAs are prepared by chemical synthesis or by in vitro transcription of different dsRNA and/or siRNA templates. In further embodiments, polypeptides with RNase III domains, including both prokaryotic and/or eukaryotic polypeptides, may be used to generate candidate siRNA molecules from double-stranded RNA. In certain embodiments, cell free extracts may also be used to generate candidate siRNA molecules in vitro. In various embodiments, in vitro transcription may include a purified linear DNA template containing a promoter, ribonucleotide triphosphates, a buffer system that includes DTT and magnesium ions, and an appropriate phage RNA polymerase, as described herein. In still further embodiments, DNA constructs with appropriate RNA polymerase promoters and dsRNA templates are prepared by standard methods and co-transfected or co-transduced to create cocktails of siRNAs in cells. Alternatively, a single DNA construct with multiple promoter/siRNA domains is transfected or transduced to create cocktails of siRNAs in cells.

A dsRNA pool or cocktail of the invention may include 2, 3, 4, 5, 6, 7, 8, 9, 10, 11, 12, 13, 14, 15, 16, 17, 18, 19, 20, or more different dsRNA molecules prepared in vitro or expressed from DNA constructs with appropriate RNA polymerase promoter and siRNA template domains. The pools of the invention may be generated by mixing or combining at least 2, 3, 4, 5, 6, 7, 8, 9, 10, 11, 12, 13, 14, 15, 16, 17, 18, 19, 20, or more different candidate siRNA molecules. A candidate siRNA molecule(s) is a dsRNA molecule(s) that may or may not have been tested for the ability to reduce gene expression of a target transcript.

In some embodiments, the invention concerns a dsRNA or siRNA that is capable of triggering RNA interference, a process by which a particular RNA sequence is destroyed (also referred to as gene silencing). siRNA are dsRNA molecules that are 100 bases or fewer in length (or have 100 basepairs or fewer in its complementarity region). A dsRNA may be 15, 16, 17, 18, 19, 20, 21, 22, 23, 24, 25, 26, 27, 28, 29, 30, 40, 50, 60, 70, 80, 90, 100 110, 120, 130, 140, 150, 160, 170, 180, 190, 200, 225, 250, 275, 300, 325, 350. 375, 400, 450, 500, 550, 600, 650, 700, 750, 800, 850, 900, 950, or 1000 nucleotides or more in length. In certain embodiments, siRNA may be approximately 21 to 25 nucleotides in length. In some cases, it has a two nucleotide 3′ overhang and a 5′ phosphate. The particular RNA sequence is targeted as a result of the complementarity between the dsRNA and the particular RNA sequence. It will be understood that dsRNA or siRNA of the invention can effect at least a 20, 30, 40, 50, 60, 70, 80, 90 percent or more reduction of expression of a targeted RNA in a cell. dsRNA of the invention (the term “dsRNA” will be understood to include “siRNA” and/or “candidate siRNA”) is distinct and distinguishable from antisense and ribozyme molecules by virtue of the ability to trigger RNAi. Structurally, dsRNA molecules for RNAi differ from antisense and ribozyme molecules in that dsRNA has at least one region of complementarity within the RNA molecule. The complementary (also referred to as “complementarity”) region comprises at least or at most 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 contiguous bases. In some embodiments, long dsRNA are employed in which “long” refers to dsRNA that are 1000 bases or longer (or 1000 basepairs or longer in complementarity region). The term “dsRNA” includes “long dsRNA”, “intermediate dsRNA” or “small dsRNA” (lengths of 2 to 100 bases or basepairs in complementarity region) unless otherwise indicated. In some embodiments of the invention, dsRNA can exclude the use of siRNA, long dsRNA, and/or “intermediate” dsRNA (lengths of 100 to 1000 bases or basepairs in complementarity region).

It is specifically contemplated that a dsRNA may be a molecule comprising two separate RNA strands in which one strand has at least one region complementary to a region on the other strand. Alternatively, a dsRNA includes a molecule that is single stranded yet has at least one complementarity region as described above (see Sui et al., 2002 and Brummelkamp et al., 2002 in which a single strand with a hairpin loop is used as a dsRNA for RNAi). For convenience, lengths of dsRNA may be referred to in terms of bases, which simply refers to the length of a single strand or in terms of basepairs, which refers to the length of the complementarity region. It is specifically contemplated that embodiments discussed herein with respect to a dsRNA comprised of two strands are contemplated for use with respect to a dsRNA comprising a single strand, and vice versa. In a two-stranded dsRNA molecule, the strand that has a sequence that is complementary to the targeted mRNA is referred to as the “antisense strand” and the strand with a sequence identical to the targeted mRNA is referred to as the “sense strand.” Similarly, with a dsRNA comprising only a single strand, it is contemplated that the “antisense region” has the sequence complementary to the targeted mRNA, while the “sense region” has the sequence identical to the targeted mRNA. Furthermore, it will be understood that sense and antisense region, like sense and antisense strands, are complementary (i.e., can specifically hybridize) to each other.

Strands or regions that are complementary may or may not be 100% complementary (“completely or fully complementary”). It is contemplated that sequences that are “complementary” include sequences that are at least 50% complementary, and may be at least 50%, 60%, 70%, 80%, or 90% complementary. In the range of 50% to 70% complementarity, such sequences may be referred to as “very complementary,” while the range of greater than 70% to less than complete complementarity can be referred to as “highly complementary.” Unless otherwise specified, sequences that are “complementary” include sequences that are “very complementary,” “highly complementary,” and “fully complementary.” It is also contemplated that any embodiment discussed herein with respect to “complementary” strands or region can be employed with specifically “fully complementary,” “highly complementary,” and/or “very complementary” strands or regions, and vice versa. Thus, it is contemplated that in some instances, as demonstrated in the Examples, that siRNA generated from sequence based on one organism may be used in a different organism to achieve RNAi of the cognate target gene. In other words, siRNA generated from a dsRNA that corresponds to a human gene may be used in a mouse cell if there is the requisite complementarity, as described above. Ultimately, the requisite threshold level of complementarity to achieve RNAi is dictated by functional capability.

It is specifically contemplated that there may be mismatches in the complementary strands or regions. Mismatches may number at most or at least 1, 2, 3, 4, 5, 6, 7, 8, 9, 10, 11, 12, 13, 14, 15, 16, 17, 18, 19, 20, 21, 22, 23, 24, 25 residues or more, depending on the length of the complementarity region.

The single RNA strand or each of two complementary double strands of a dsRNA molecule may be of at least or at most the following lengths: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 31, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 or more (including the full-length of a particular's gene's mRNA without the poly-A tail) bases or basepairs. If the dsRNA is composed of two separate strands, the two strands may be the same length or different lengths. If the dsRNA is a single strand, in addition to the complementarity region, the strand may have 1, 2, 3, 4, 5, 6, 7, 8, 9, 10, 11, 12, 13, 14, 15, 16, 17, 18, 19, 20, 21, 22, 23, 24, 25, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more bases on either or both ends (5′ and/or 3′) or as forming a hairpin loop between the complementarity regions.

In some embodiments, the strand or strands of dsRNA are 100 bases (or basepairs) or less, in which case they may also be referred to as candidate “siRNA.” In specific embodiments the strand or strands of the dsRNA are less than 70 bases in length. With respect to those embodiments, the dsRNA strand or strands may be from 5-70, 10-65, 20-60, 30-55, 40-50 bases or basepairs in length. A dsRNA that has a complementarity region equal to or less than 30 basepairs (such as a single stranded hairpin RNA in which the stem or complementary portion is less than or equal to 30 basepairs) or one in which the strands are 30 bases or fewer in length is specifically contemplated, as such molecules evade a mammalian's cell antiviral response. Thus, a hairpin dsRNA (one strand) may be 70 or fewer bases in length with a complementary region of 30 basepairs or fewer. In some cases, a dsRNA may be processed in the cell into siRNA.

Methods and compositions, including kits, of the invention concern RNase III, which is an enzyme that cleaves double stranded RNA into one or more pieces that are 12-30 base pairs in length, or 12-15 basepairs or 20-23 basepairs in length in some embodiments Thus, candidate siRNA molecules (which refers to dsRNA that are the appropriate length to mediate or trigger RNAi, but it is not yet known whether it can achieve RNAi) may be 12, 13, 14, 15, 16, 17, 18, 19, 20, 21, 22, 23, 24, 25, 26, 27, 28, 29, or 30 basepairs in length.

Furthermore, it is contemplated that siRNA or the longer dsRNA template may be labeled. The label may be fluorescent, radioactive, enzymatic, or colorimetric.

The substrate for RNase III of the invention is a dsRNA molecule, which may be composed of two strands or a single strand with a region of complementarity within the strand. It is contemplated that the dsRNA substrate may be 25 to 10,000, 25 to 5,000, 50 to 1,000, 100-500, or 100-200 nucleotides or basepairs in length. Alternatively the dsRNA substrate may be at least or at most 25, 50, 75, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5500, 6000, 6500, 7000, 7500, 8000, 8500, 9000, 9500, or 10,000 or more nucleotides of basepairs in length. dsRNA need only correspond to part of the target gene to yield an appropriate siRNA. Thus, a dsRNA that corresponds to all or part of a target gene means that the dsRNA can be cleaved to yield at least one siRNA that can silence the target gene. The dsRNA may contain sequences that do not correspond to the target gene, or the dsRNA may contain sequences that correspond to multiple target genes.

The invention also concerns labeled dsRNA. It is contemplated that a dsRNA may have one label attached to it or it may have more than one label attached to it. When more than one label is attached to a dsRNA, the labels may be the same or be different. If the labels are different, they may appear as different colors when visualized. The label may be on at least one end and/or it may be internal. Furthermore, there may be a label on each end of a single stranded molecule or on each end of a dsRNA made of two separate strands. The end may be the 3′ and/or the 5′ end of the nucleic acid. A label may be on the sense strand or the sense end of a single strand (end that is closer to sense region as opposed to antisense region), or it may be on the antisense strand or antisense end of a single strand (end that is closer to antisense region as opposed to sense region). In some cases, a strand is labeled on a particular nucleotide (G, A, U, or C).

When two or more differentially colored labels are employed, fluorescent resonance energy transfer (FRET) techniques may be employed to characterize the dsRNA.

Labels contemplated for use in several embodiments are non-radioactive. In many embodiments of the invention, the labels are fluorescent, though they may be enzymatic, radioactive, or positron emitters. Fluorescent labels that may be used include, but are not limited to, BODIPY, Alexa Fluor, fluorescein, Oregon Green, tetramethylrhodamine, Texas Red, rhodamine, cyanine dye, or derivatives thereof. The labels may also more specifically be Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5, DAPI, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, TET, Tetramethylrhodamine, and/or Texas Red. A labeling reagent is a composition that comprises a label and that can be incubated with the nucleic acid to effect labeling of the nucleic acid under appropriate conditions. In some embodiments, the labeling reagent comprises an alkylating agent and a dye, such as a fluorescent dye. In some embodiments, a labeling reagent comprises an alkylating agent and a fluorescent dye such as Cy3, Cy5, or fluorescein (FAM). In still further embodiments, the labeling reagent is also incubated with a labeling buffer, which may be any buffer compatible with physiological function (i.e., buffers that is not toxic or harmful to a cell or cell component) (termed “physiological buffer”).

In some embodiments of the invention, a dsRNA has one or more non-natural nucleotides, such as a modified residue or a derivative or analog of a natural nucleotide. Any modified residue, derivative or analog may be used to the extent that it does not eliminate or substantially reduce (by at least 50%) RNAi activity of the dsRNA.

A person of ordinary skill in the art is well aware of achieving hybridization of complementary regions or molecules. Such methods typically involve heat and slow cooling of temperature during incubation.

Any cell that undergoes RNAi can be employed in methods of the invention. The cell may be a eukaryotic cell, mammalian cell such as a primate, rodent, rabbit, or human cell, a prokaryotic cell, or a plant cell. In some embodiments, the cell is alive, while in others the cell or cells is in an organism or tissue. Alternatively, the cell may be dead. The dead cell may also be fixed. In some cases, the cell is attached to a solid, non-reactive support such as a plate or petri dish. Such cells may be used for array analysis. It is contemplated that cells may be grown on an array and dsRNA administered to the cells.

In some embodiments of the invention, there are methods of reducing the expression of a target gene in a cell. Such methods involve the compositions described above, including the embodiments described for RNase III, dsRNA, and siRNA.

In various embodiments of the invention, reduction or elimination of expression of at least 1, 2, 3, 4, 5, or more target genes may be accomplished by the a) obtaining at least two dsRNA molecules corresponding one or more target genes and b) transfecting the dsRNA molecules corresponding to the one or more target gene into a cell. The dsRNA molecules may be candidate or confirmed siRNA molecules. The methods of the invention may include 2, 3, 4, 5, 6, 7, 8, 9, 10, 11, 12, 13, 14, 15, 16, 17, 18, 19, 20, 25, 30, 35 or more dsRNA molecules corresponding to at least one or more target genes.

Methods of creating dsRNA molecules or pools of candidate siRNAs may use the methods described herein including, but not limited to methods involving a) obtaining a dsRNA that corresponds to at least 15 contiguous basepairs of at least a first target gene b) incubating a dsRNA corresponding to part of at least one target gene with an effective amount of composition comprising RNase III under conditions to allow RNase III to cleave the dsRNA into siRNA; and/or c) transfecting the siRNA into the cell. The term “effective amount” in the context of RNase III refers to an amount that will effect cleavage of a dsRNA substrate by RNase III. “Target gene” or “targeted gene” refers to a gene whose expression is desired to be reduced, inhibited or eliminated through RNA interference. RNA interference directed to a target gene requires an siRNA that is complementary in one strand and identical in the other strand to a portion of the coding region of the targeted gene.

In additional methods of the invention, one or more dsRNA may be the substrate for RNase III activity, but only some of the resulting products are characterized as siRNA because not all of the products can effect RNAi. The products of dsRNA cleavage by RNase III are candidate siRNAs. By processing a long dsRNA into a pool of dsRNA, the need for determining which RNA product is an siRNA is rendered moot or diminished.

Further embodiments of the invention concern generating candidate siRNA to trigger RNAi in a cell to a target gene. Any of the methods described herein for reducing the expression of a target gene can be applied to generating candidate siRNA and vice versa. Furthermore, it is specifically contemplated that the generation of candidate siRNA from a longer dsRNA molecule may be done outside of a cell (in vitro). In fact, particular embodiments of the invention take advantage of the benefits of employing compositions that can be manipulated in a test tube, as opposed to in a cell.

In additional methods of the invention at least one siRNA molecule is isolated away from the other siRNA molecules. However, it is specifically contemplated that all or a subset of the candidate siRNA products that result from RNase III cleavage(s) may be employed in methods of the invention. Thus, pools of candidate siRNAs directed to a single or multiple targets may be transfected or administered to a cell to trigger RNAi against the target(s).

In some methods of the invention, siRNA and/or candidate siRNA molecules or template nucleic acids may be isolated or purified prior to their being used in a subsequent step. siRNA and/or candidate siRNA molecules may be isolated or purified prior to introduction into a cell. “Introduction” into a cell includes known methods of transfection, transduction, infection and other methods for introducing an expression vector or a heterologous nucleic acid into a cell. A template nucleic acid or amplification primer may be isolated or purified prior to it being transcribed or amplified. Isolation or purification can be performed by a number of methods known to those of skill in the art with respect to nucleic acids. In some embodiments, a gel, such as an agarose or acrylamide gel, is employed to isolate the siRNA and/or candidate siRNA.

In some methods of the invention dsRNA is obtained by transcribing each strand of the dsRNA from one or more cDNA (or DNA or RNA) encoding the strands in vitro. It is contemplated that a single template nucleic acid molecule may be used to transcribe a single RNA strand that has at least one region of complementarity (and is thus double-stranded under conditions of hybridization) or it may be used to transcribe two separate complementary RNA molecules. Alternatively, more than one template nucleic acid molecule may be transcribed to generate two separate RNA strands that are complementary to one another and capable of forming a dsRNA.

Additional methods involve isolating the transcribed strand(s) and/or incubating the strand(s) under conditions that allow the strand(s) to hybridize to their complementary strands (or regions if a single strand is employed).

Nucleic acid templates may be generated by a number of methods well known to those of skill in the art. In some embodiments the template, such as a cDNA, is synthesized through amplification or it may be a nucleic acid segment in or from a plasmid that harbors the template.

In various embodiments, siRNAs are encoded by expression constructs. The expression constructs may be obtained and introduced into a cell. Once introduced into the cell the expression construct is transcribed to produce various siRNAs. Expression constructs include nucleic acids that provide for the transcription of a particular nucleic acid. Expression constructs include plasmid DNA, linear expression elements, circular expression elements, viral expression constructs, and the like, all of which are contemplated as being used in the compositions and methods of the present invention. In certain embodiments at least 2, 3, 4, 5, 6, 7, 8, 9, 10 or more siRNA molecules are encoded by a single expression construct. Expression of the siRNA molecules may be independently controlled by at least 2, 3, 4, 5, 6, 7, 8, 9, 10 or more promoter elements. In certain embodiments, at least 2, 3, 4, 5, 6, 7, 8, 9, 10 or more expression constructs may introduced into the cell. Each expression construct may encode 1, 2, 3, 4, 5, 6, 7, 8, 9, 10 or more siRNA molecules. In certain embodiments siRNA molecules may be encoded as expression domains. Expression domains include a transcription control element, which may or may not be independent of other control or promoter elements; a nucleic acid encoding an siRNA; and optionally a transcriptional termination element. In other words, an siRNA cocktail or pool may be encoded by a single or multiple expression constructs. In particular embodiments the expression construct is a plasmid expression construct.

Other methods of the invention also concern transcribing a strand or strands of a dsRNA using a promoter that can be employed in vitro or outside a cell, such as a prokaryotic promoter. In some embodiments, the prokaryotic promoter is a bacterial promoter or a bacteriophage promoter. It is specifically contemplated that dsRNA strands are transcribed with SP6, T3, or T7 polymerase.

Methods for generating siRNA or candidate siRNA to more than one target gene are considered part of the invention. Thus, siRNA or candidate siRNA directed to 1, 2, 3, 4, 5, 6, 7, 8, 9, 10 or more target genes may be generated and implemented in methods of the invention. An array can be created with pools of siRNA and/or candidate siRNA to multiple targets may be used as part of the invention.

In specific embodiments of the invention, there are methods for achieving RNA interference of a target gene in a cell using one or more siRNA molecules. These methods involve: a) generating at least one double-stranded DNA template (which may comprise an SP6, T3, or T7 promoter on at least one strand) corresponding to part of the target gene; b) transcribing the template, wherein either i) a single RNA strand with a complementarity region is created or ii) first and second complementary RNA strands are created; c) hybridizing either the single complementary RNA strand or the first and second complementary RNA strands to create a dsRNA molecule corresponding to the target gene; d) incubating the dsRNA molecule with a polypeptide comprising an RNase III domain, under conditions to allow cleavage of the dsRNA into at least two candidate siRNA molecules; and, e) transfecting at least one siRNA into the cell.

In some methods of the invention, a candidate siRNA may be tested for its ability to mediate or trigger RNAi, however, in some embodiments of the invention, it is not assayed. Instead, multiple siRNAs directed to different portions of the same target may be employed to reduce expression of the target.

It is specifically contemplated that any method of the invention may be employed with any kit component or composition described herein. Furthermore, any kit may contain any component described herein and any component involved in any method of the invention. Thus, any element discussed with respect to one embodiment may be applied to any other embodiment of the invention.

The present invention concerns preparing cocktails of siRNAs or DNA constructs capable of expressing cocktails of siRNAs that target RNAs that might be present in cells. The siRNA cocktails or DNA constructs expressing cocktails of siRNAs can be co-transfected or co-transduced to provide for the specific reduction in the levels of the target RNA. The present invention also concerns kits that can be used to generate siRNA and siRNA candidate molecules. Additionally, the present invention also concerns kits that provide a cocktail or pool of siRNAs or DNA constructs capable of expressing cocktails of siRNAs that target RNAs that might be present in cells directed to a particular nucleic acid, gene, or combination of genes. In some embodiments, the cocktails may be provided as combinations of 2, 3, 4, 5, 6, 7, 8, 9, 10 or more siRNAs or DNA constructs capable of expressing cocktails of siRNAs that target RNAs that might be present in cells in 1, 2, 3, 4, 5, 6, 7, 8, 9, 10 or more packages in a kit. In some embodiments, 1, 2, 3, 4, 5, 6, 7, 8, 9, 10 or more cocktails may be provided in one or more kits. Components of the kit may be provided in concentrations of about 1×, 2×, 3×, 4×, 5×, 6×, 7×, 8×, 9×, 10×, 15×, 20×, 25× or higher with respect to final reaction volumes. Such concentrations apply specifically with respect to buffers in the kit. Kits of the invention may also include reagents for the introduction of the cocktails into a cell, e.g., transfection reagents.

All methods of the invention may use kit embodiments to achieve a method of reducing the expression of a target gene in a cell or for simply generating an siRNA or a candidate siRNA.

The present invention also concerns kits for labeling and using dsRNA for RNA interference. Kits may comprise components, which may be individually packaged or placed in a container, such as a tube, bottle, vial, syringe, or other suitable container means. Kit embodiments include the one of more of the following components: labeling buffer comprising a physiological buffer with a pH range of 7.0 to 7.5; labeling reagent for labeling dsRNA with fluorescent label comprising an alkylating agent; control dsRNA comprising a dsRNA known to trigger RNAi in a cell, such as those disclosed herein, nuclease free water, ethanol, NaCl, reconstitution solution comprising DMSO or annealing buffer comprising Hepes and at least one salt. In further embodiments, the labeling reagent comprises Cy3, Cy5, and/or fluorescein (FAM).

Individual components may also be provided in a kit in concentrated amounts; in some embodiments, a component is provided individually in the same concentration as it would be in a solution with other components. Concentrations of components may be provided as 1×, 2×, 5×, 10×, 15×, or 20× or more.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF ILLUSTRATIVE EMBODIMENTS

The present invention is directed to compositions and methods relating to a mixture or pool of double stranded RNA molecules that can be used in the process of RNA interference (RNAi). RNAi results in a reduction of expression of one or more target gene(s). Double stranded RNA has been shown to reduce gene expression of a target. A portion of one strand of the double stranded RNA is complementary to a region of the target's mRNA while another portion of the double stranded RNA molecule is identical to the same region of the target's mRNA. As discussed earlier, the RNA molecule of the invention is double stranded, which may be accomplished through two separate strands or a single strand having one region complementary to another region of the same strand. Exemplary methods for siRNA production may be found in U.S. Provisional Application Ser. No. 60/353,332, which is hereby incorporated by reference. Discussed below are uses for the present invention—compositions, methods, and kits—and ways of implementing the invention.

Various embodiments of the invention include processes where such double stranded RNA molecules, such as siRNAs, candidate siRNAs or dsRNAs, may be generated to one or more target genes and a 75% or greater reduction in the abundance of the gene product in approximately 95% of the cases may be observed. Furthermore, at least a 50% reduction in target gene expression was observed in approximately every case studied. The processes typically rely on the co-transfection of multiple siRNAs or candidate siRNAs to the same target gene, i.e. dsRNA pools. Multiple siRNAs or candidate siRNAs may be co-transfected without causing any non-specific effects in the transfected cells. Furthermore, contrary to published reports, co-transfecting multiple siRNAs does not limit the activity of any given siRNA. Rather, additive effects among the siRNAs or candidate siRNAs were observed. For instance, if four siRNAs are transfected wherein one siRNA reduces gene expression by 80%, two by 50%, and one not at all, typically a 90-95% reduction in target gene expression is seen.

The mixture of dsRNAs, candidate siRNA or siRNAs is referred to as an siRNA cocktail or dsRNA pool. The term “cocktail” is used interchangeably with the term “pool” throughout this application. In addition to improving the success rate for siRNA experiments in a given cell line, the methods described may improve methods that involve multiple cell lines. Different cell lines may respond differently to a given siRNA or candidate siRNA. For instance, a particular first siRNA that provides a 90% reduction in the expression of a given target gene in a first cell line might not be at all effective in a second cell line. siRNA cocktails or pools reduce or eliminate this problem by covering target sequences over a greater number of cell lines.

At least two general methods for preparing siRNA cocktails or pools are typically employed. In the first, multiple siRNA target sites are identified in a given gene. Two or more of these are selected for siRNA or candidate siRNA preparation. The siRNAs may be generated either by chemical synthesis using standard procedures or by in vitro transcription from DNA templates. Equal or non-equal amounts of the siRNAs can be mixed to prepare siRNA cocktails or pools for transfection.

In another method, long dsRNAs are prepared, typically by in vitro transcription. Long dsRNAs bearing sequence to at least one target gene are converted to siRNAs or candidate siRNAs by the action of a double strand RNA specific nuclease such as RNAse III or Dicer. The resulting siRNAs may be derived from different regions of the original dsRNA, providing multiple unique siRNAs or candidate siRNAs specific to at least one region or domain in at least one target gene.

Alternatively, DNA constructs with RNA polymerase promoters and siRNA template sequences can be prepared and introduced to cells wherein siRNA cocktails are expressed. The different siRNAs can either be expressed from multiple DNA constructs or from a single DNA molecules with multiple siRNA expression domains.

Candidate siRNA or siRNA cocktails or pools have been found to significantly reduce the time required for siRNA development. In fact, candidate siRNA cocktails or pools may eliminate the need to measure the reduction in gene expression because most every cocktail or pool may reduce the target gene expression by approximately 50-95%.

Given that siRNAs or candidate siRNA pools that work effectively in greater than 50% of the cases may be produced and that siRNAs function independently, design or production of combinations of siRNAs or candidate siRNA may reduce the expression of target genes by greater than 75% are typically produced with a reasonable certainty. For instance, if it is assumed that 50% of optimally-designed siRNAs reduce gene expression by at least 75%, then designing or producing a pool of four siRNAs to a single target and co-transfecting them should provide an almost 95% chance (1-(½)⁴) that the expression of the targeted gene will be reduced by 75%. Furthermore, a majority of the siRNA or candidate siRNA pools will typically provide at least a 50% reduction in gene expression. The transfection of siRNA or candidate siRNA pools or mixtures to may reduce or eliminate the need to validate all siRNAs as the vast majority of target genes will typically be reduced to levels that result in a biological effect. This technique may be used to develop siRNA or candidate siRNA pools or mixtures to related sets of genes to facilitate functional screening assays.

Therefore, a method in which a mixture of siRNA can be made from a single reaction would increase the likelihood of knocking down the gene the first time it is performed.

### I. RNA Interference (RNAi)

RNA interference (also referred to as “RNA-mediated interference”)(RNAi) is a mechanism by which gene expression can be reduced or eliminated. Double stranded RNA (dsRNA) or single stranded RNA has been observed to mediate the reduction, which is a multi-step process (for details of single stranded RNA methods and compositions see Martinez et al., 2002). dsRNA activates post-transcriptional gene expression surveillance mechanisms that appear to function to defend cells from virus infection and transposon activity (Fire et al., 1998; Grishok et al., 2000; Ketting et al., 1999; Lin et al., 1999; Montgomery et al., 1998; Sharp et al., 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene. (Fire et al., 1998; Grishok et al., 2000; Ketting et al., 1999; Lin et al., 1999; Montgomery et al., 1998; Sharp, 1999; Sharp et al., 2000; Tabara et al., 1999). Moreover, dsRNA has been shown to silence genes in a wide range of systems, including plants, protozoans, fungi, C. elegans, Trypanasoma, Drosophila, and mammals (Grishok et al., 2000; Sharp, 1999; Sharp et al., 2000; Elbashir et al., 2001).

Some of the uses for RNAi include identifying genes that are essential for a particular biological pathway, identifying disease-causing genes, studying structure function relationships, and implementing therapeutics and diagnostics. As with other types of gene inhibitory compounds, such as antisense and triplex forming oligonucleotides, tracking these potential drugs in vivo and in vitro is important for drug development, pharmacokinetics, biodistribution, macro and microimaging metabolism and for gaining a basic understanding of how these compounds behave and function. siRNAs have high specificity and may perhaps be used to knock out the expression of a single allele of a dominantly mutated diseased gene.

A. Polypeptides with RNAse III Domains

In certain embodiments, the present invention concerns compositions comprising at least one proteinaceous molecule, such as RNase III, DICER or a polypeptide having RNase III activity or an RNase III domain. Exemplary methods and compositions may be found in U.S. Provisional Application Ser. No. 60/402,347, which is hereby incorported by reference.

In further embodiments of the invention, RNase III is from a prokaryote, including a gram negative bacteria. Thus, the present invention may refer to a “non-eukaryotic RNase III” to exclude eukaryotic-derived proteins such as Dicer or it may refer to “prokaryotic RNase III” to refer to an RNase III protein derived from a prokaryotic organism. In additional embodiments of the invention, the RNase III is from E. coli, a gram-negative bacteria. The RNase III from E. coli may have the amino acid sequence of GenBank Accession Number NP_{—}289124 (SEQ ID NO:1), which is specifically incorporated by reference.

In various embodiments of the invention, methods and compositions involve a protein or polypeptide with RNase III activity (that is, the ability to cleave double stranded RNA into smaller segments) or a protein or polypeptide with an RNase III domain. An “RNase III domain” refers to an amino acid region that confers the ability to cleave double stranded RNA into smaller segments, and which is understood by those of skill in the art and as described elsewhere herein.

In other compositions and methods of the invention, the RNase III may be purified from an organism's endogenous supply of RNase III; alternatively, recombinant RNase III may be purified from a cell or an in vitro expression system. The term “recombinant” refers to a compound that is produced by from a nucleic acid (or a replicated version thereof) that has been manipulated in vitro, for example, being digested with a restriction endonuclease, cloned into a vector, amplified, etc. The terms “recombinant RNase III” and “recombinantly produced RNase III” refer to an active RNase III polypeptide that was prepared from a nucleic acid that was manipulated in vitro or is the replicated version of such a nucleic acid. It is specifically contemplated that RNase III may be recombinantly produced in a prokaryotic or eukaryotic cell. It may be produced in a mammalian cell, a bacterial cell, a yeast cell, or an insect cell. In specific embodiments of the invention, the RNase III is produced from a baculovirus expression system involving insect cells. Alternatively, recombinant RNase III may be produced in vitro or it may be chemically synthesized. Such RNase III may first be purified for use in RNA interference. Purification may allow the RNAse III to retain activity in concentrations of about 0.1, 0.2, 0.3, 0.4, 0.5, 0.6, 0.7, 0.8, 0.9, 1, 2, 3, 4, 5, 6, 7, 8, 9, 10 or more units/microliter. A “unit” is defined as the amount of enzyme that digests 1 μg of a 500 basepair dsRNA in 60 minutes at 37° C. into RNA products that are 12-15 basepairs in length.

It is contemplated that the use of the term “about” in the context of the present invention is to connote inherent problems with precise measurement of a specific element, characteristic, or other trait. Thus, the term “about,” as used herein in the context of the claimed invention, simply refers to an amount or measurement that takes into account single or collective calibration and other standardized errors generally associated with determining that amount or measurement. For example, a concentration of “about” 100 mM of Tris can encompass an amount of 100 mM±5 mM, if 5 mM represents the collective error bars in arriving at that concentration. Thus, any measurement or amount referred to in this application can be used with the term “about” if that measurement or amount is susceptible to errors associated with calibration or measuring equipment, such as a scale, pipetteman, pipette, graduated cylinder, etc.

RNase III polypeptides or polypeptides with an RNase III domain or activity may be used in conjunction with an enzyme dilution buffer. In some embodiments, the composition comprises an enzyme dilution buffer. The enzymes of the invention may be provided in such a buffer. In some embodiments, the buffer comprises one or more of the following glycerol, Tris, dithiothreitol (DTT), or EDTA. In specific embodiments, the enzyme dilution buffer comprises 50% glycerol, 20 mM Tris, 0.5 mM DTT, and 0.5 mM EDTA. In a method employing a composition, these components of the buffer may be diluted after addition of other components to the composition.

In still further embodiments of the invention, recombinantly produced RNase III may be truncated by or be missing 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more contiguous amino acids in one or more places in the polypeptide, yet still retain RNase III activity. In addition or alternatively, an RNase III polypeptide may include a heterologous sequence of at least 3 amino acids and also still retain RNase III activity. The heterologous sequence may be a discernible region (contiguous stretch of amino acids) from another polypeptide to render the RNase III polypeptide chimeric. The heterologous sequence may be tag that facilitates production or purification of the RNase III. Thus, in some embodiments of the invention, recombinant RNase III has a tag attached to it, either on one of its ends or attached at any residue in between. In some embodiments the tag is a histidine tag (His-tag), which is a series of at least 3 histidine residues and in some embodiments, 4, 5, 6, 7, 8, 9, 10, or more consecutive histidine residues. In other embodiments, the tag is GST, streptavidin, or FLAG. Additionally, some RNase III polypeptides may have a tag initially, but the tag may be removed subsequently.

As used herein, a “proteinaceous molecule,” “proteinaceous composition,” “proteinaceous compound,” “proteinaceous chain” or “proteinaceous material” generally refers, but is not limited to, a protein of greater than about 200 amino acids or the full length endogenous sequence translated from a gene; a polypeptide of greater than about 100 amino acids; and/or a peptide of from 3 to 100 amino acids. All the “proteinaceous” terms described above may be used interchangeably herein.

In certain embodiments the size of the at least one proteinaceous molecule may comprise, but is not limited to 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 or greater amino molecule residues, and any range derivable therein.

Accordingly, the term “proteinaceous composition” encompasses amino molecule sequences comprising at least one of the 20 common amino acids in naturally synthesized proteins, or at least one modified or unusual amino acid, including but not limited to those shown on Table 1 below.

TABLE 1
Modified and Unusual Amino Acids

| Abbr. | Amino Acid | Abbr. | Amino Acid |
|---|---|---|---|
| Aad | 2-Aminoadipic acid | EtAsn | N-Ethylasparagine |
| Baad | 3-Aminoadipic acid | Hyl | Hydroxylysine |
| Bala | β-alanine, β-Amino- | AHyl | allo-Hydroxylysine |
| | propionic acid | | |
| Abu | 2-Aminobutyric acid | 3Hyp | 3-Hydroxyproline |
| 4Abu | 4-Aminobutyric acid, | 4Hyp | 4-Hydroxyproline |
| | piperidinic acid | | |
| Acp | 6-Aminocaproic acid | Ide | Isodesmosine |
| Ahe | 2-Aminoheptanoic acid | AIle | allo-Isoleucine |
| Aib | 2-Aminoisobutyric acid | MeGly | N-Methylglycine, |
| | | | sarcosine |
| Baib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

In certain embodiments the proteinaceous composition comprises at least one protein, polypeptide or peptide. In further embodiments the proteinaceous composition comprises a biocompatible protein, polypeptide or peptide. As used herein, the term “biocompatible” refers to a substance which produces no significant untoward effects when applied to, or administered to, a given organism according to the methods and amounts described herein. Such untoward or undesirable effects are those such as significant toxicity or adverse immunological reactions. In preferred embodiments, biocompatible protein, polypeptide or peptide containing compositions will generally be mammalian proteins or peptides or synthetic proteins or peptides each essentially free from toxins, pathogens and harmful immunogens.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. The nucleotide and protein, polypeptide and peptide sequences for various genes have been previously disclosed, and may be found at computerized databases known to those of ordinary skill in the art. One such database is the National Center for Biotechnology Information's Genbank and GenPept databases (http://www.ncbi.nlm.nih.gov/). The coding regions for these known genes may be amplified and/or expressed using the techniques disclosed herein or as would be know to those of ordinary skill in the art. Alternatively, various commercial preparations of proteins, polypeptides and peptides are known to those of skill in the art.

In certain embodiments a proteinaceous compound may be purified. Generally, “purified” will refer to a specific protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

It is contemplated that virtually any protein, polypeptide or peptide containing component may be used in the compositions and methods disclosed herein. However, it is preferred that the proteinaceous material is biocompatible. In certain embodiments, it is envisioned that the formation of a more viscous composition will be advantageous in that will allow the composition to be more precisely or easily applied to the tissue and to be maintained in contact with the tissue throughout the procedure. In such cases, the use of a peptide composition, or more preferably, a polypeptide or protein composition, is contemplated. Ranges of viscosity include, but are not limited to, about 40 to about 100 poise. In certain aspects, a viscosity of about 80 to about 100 poise is preferred.

1. Functional Aspects

When the present application refers to the function or activity of RNase III, it is meant that the molecule in question has the ability to cleave a double-stranded RNA substrate into one or more dsRNA products.

2. Variants of RNase III and Proteins with RNase III Activity

Amino acid sequence variants of the polypeptides of the present invention can be substitutional, insertional or deletion variants. Deletion variants lack one or more residues of the native protein that are not essential for function or immunogenic activity, and are exemplified by the variants lacking a transmembrane sequence described above. Another common type of deletion variant is one lacking secretory signal sequences or signal sequences directing a protein to bind to a particular part of a cell. Insertional mutants typically involve the addition of material at a non-terminal point in the polypeptide. This may include the insertion of a single residue. Terminal additions, called fusion proteins, are discussed below.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The term “biologically functional equivalent” is well understood in the art and is further defined in detail herein. Accordingly, sequences that have between about 70% and about 80%; or more preferably, between about 81% and about 90%; or even more preferably, between about 91% and about 99%; of amino acids that are identical or functionally equivalent to the amino acids of an RNase III polypeptide or a protein having an RNase III domain, provided the biological activity of the protein is maintained.

The term “functionally equivalent codon” is used herein to refer to codons that encode the same amino acid, such as the six codons for arginine or serine, and also refers to codons that encode biologically equivalent amino acids (see Table 2, below).

TABLE 2
Codon Table

| Amino Acids | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

It also will be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity, as discussed below. Table 2 shows the codons that encode particular amino acids.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine *−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still produce a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

Another embodiment for the preparation of polypeptides according to the invention is the use of peptide mimetics. Mimetics are peptide-containing molecules that mimic elements of protein secondary structure. See e.g., Johnson (1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the natural properties of RNase III or a protein with an RNase III domain, but with altered and even improved characteristics.

3. Fusion Proteins

A specialized kind of insertional variant is the fusion protein. This molecule generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes such as a hydrolase, glycosylation domains, cellular targeting signals or transmembrane regions.

4. Protein Purification

It may be desirable to purify RNase III, a protein with an RNase domain, or variants thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques, including a Nickel column or using Histidine or glutathione tags. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

B. Nucleic Acids for RNAi

The present invention concerns double-stranded RNA that may or may not be capable of triggering RNAi. The RNA may be synthesized chemically or it may be produced recombinantly. They may be subsequently isolated and/or purified.

As used herein, the term “dsRNA” refers to a double-stranded RNA molecule and includes or is synonymous with candidate siRNA. The molecule may be a single strand with intra-strand complementarity such that two portions of the strand hybridize with each other or the molecule may be two separate RNA strands that are partially or fully complementary to each other along one or more regions or along their entire lengths. Partially complementary means the regions are less than 100% complementary to each other, but that they are at least 50%, 60%, 70%, 80%, or 90% complementary to each other.

The siRNA and/or candidate siRNA cocktails described in the present invention allows for the modulation and especially the attenuation of target gene expression when such a gene is present and liable to expression within a cell. Modulation of expression can be partial or complete inhibition of gene function, or even the up-regulation of other, secondary target genes or the enhancement of expression of such genes in response to the inhibition of the primary target gene. Attenuation of gene expression may include the partial or complete suppression or inhibition of gene function, transcript processing or translation of the transcript. In the context of RNA interference, modulation of gene expression is thought to proceed through a complex of proteins and RNA, specifically including small, dsRNA that may act as a “guide” RNA. The siRNA therefore is thought to be effective when its nucleotide sequence sufficiently corresponds to at least part of the nucleotide sequence of the target gene. Although the present invention is not limited by this mechanistic hypothesis, it is highly preferred that the sequence of nucleotides in the siRNA be substantially identical to at least a portion of the target gene sequence.

A target gene generally means a polynucleotide comprising a region that encodes a polypeptide, or a polynucleotide region that regulates replication, transcription or translation or other processes important tot expression of the polypeptide, or a polynucleotide comprising both a region that encodes a polypeptide and a region operably linked thereto that regulates expression. The targeted gene can be chromosomal (genomic) or extrachromosomal. It may be endogenous to the cell, or it may be a foreign gene (a transgene). The foreign gene can be integrated into the host genome, or it may be present on an extrachromosomal genetic construct such as a plasmid or a cosmid. The targeted gene can also be derived from a pathogen, such as a virus, bacterium, fungus or protozoan, which is capable of infecting an organism or cell. Target genes may be viral and pro-viral genes that do not elicit the interferon response, such as retroviral genes. The target gene may be a protein-coding gene or a non-protein coding gene, such as a gene which codes for ribosomal RNAs, splicosomal RNA, tRNAs, etc.

Any gene being expressed in a cell can be targeted. Preferably, a target gene is one involved in or associated with the progression of cellular activities important to disease or of particular interest as a research object. Thus, by way of example, the following are classes of possible target genes that may be used in the methods of the present invention to modulate or attenuate target gene expression: developmental genes (e.g. adhesion molecules, cyclin kinase inhibitors, Wnt family members, Pax family members, Winged helix family members, Hox family members, cytokines/lymphokines and their receptors, growth or differentiation factors and their receptors, neurotransmitters and their receptors), oncogenes (e.g. ABLI, BLC1, BCL6, CBFA1, CBL, CSFIR, ERBA, ERBB, EBRB2, ETS1, ETS1, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3 and YES), tumor suppresser genes (e.g. APC, BRCA1, BRCA2, MADH4, MCC, NF1, NF2, RB1, TP53 and WT1), and enzymes (e.g. ACP desaturases and hydroxylases, ADP-glucose pyrophorylases, ATPases, alcohol dehydrogenases, amylases, amyloglucosidases, catalases, cellulases, cyclooxygenases, decarboxylases, dextrinases, esterases, DNA and RNA polymerases, galactosidases, glucanases, glucose oxidases, GTPases, helicases, hemicellulases, integrases, invertases, isomersases, kinases, lactases, lipases, lipoxygenases, lysozymes, pectinesterases, peroxidases, phosphatases, phospholipases, phosphorylases, polygalacturonases, proteinases and peptideases, pullanases, recombinases, reverse transcriptases, topoisomerases, xylanases).

The nucleotide sequence of the siRNA is defined by the nucleotide sequence of its target gene. The siRNA contains a nucleotide sequence that is essentially identical to at least a portion of the target gene. Preferably, the siRNA contains a nucleotide sequence that is completely identical to at least a portion of the target gene. Of course, when comparing an RNA sequence to a DNA sequence, an “identical” RNA sequence will contain ribonucleotides where the DNA sequence contains deoxyribonucleotides, and further that the RNA sequence will typically contain a uracil at positions where the DNA sequence contains thymidine.

A siRNA comprises a double stranded structure, the sequence of which is “substantially identical” to at least a portion of the target gene. “Identity,” as known in the art, is the relationship between two or more polynucleotide (or polypeptide) sequences, as determined by comparing the sequences. In the art, identity also means the degree of sequence relatedness between polynucleotide sequences, as determined by the match of the order of nucleotides between such sequences. Identity can be readily calculated. See, for example: Computational Molecular Biology, Lesk, A. M., ed. Oxford University Press, New York, 1988; Biocomputing: Informatics and Genome Projects, Smith, D. W., ed., Academic Press, New York, 1993; and the methods disclosed in WO 99/32619, WO 01/68836, WO 00/44914, and WO 01/36646, specifically incorporated herein by reference. While a number of methods exist for measuring identity between two nucleotide sequences, the term is well known in the art. Methods for determining identity are typically designed to produce the greatest degree of matching of nucleotide sequence and are also typically embodied in computer programs. Such programs are readily available to those in the relevant art. For example, the GCG program package (Devereux et al.), BLASTP, BLASTN, and FASTA (Atschul et al.) and CLUSTAL (Higgins et al., 1992; Thompson, et al., 1994).

One of skill in the art will appreciate that two polynucleotides of different lengths may be compared over the entire length of the longer fragment. Alternatively, small regions may be compared. Normally sequences of the same length are compared for a final estimation of their utility in the practice of the present invention. It is preferred that there be 100% sequence identity between the dsRNA for use as siRNA and at least 15 contiguous nucleotides of the target gene, although a dsRNA having 70%, 75%, 80%, 85%, 90%, or 95% or greater may also be used in the present invention. A siRNA that is essentially identical to a least a portion of the target gene may also be a dsRNA wherein one of the two complementary strands (or, in the case of a self-complementary RNA, one of the two self-complementary portions) is either identical to the sequence of that portion or the target gene or contains one or more insertions, deletions or single point mutations relative to the nucleotide sequence of that portion of the target gene. siRNA technology thus has the property of being able to tolerate sequence variations that might be expected to result from genetic mutation, strain polymorphism, or evolutionary divergence.

RNA (ribonucleic acid) is known to be the transcription product of a molecule of DNA (deoxyribonucleic acid) synthesized under the action of an enzyme, DNA-dependent RNA polymerase. There are diverse applications of the obtaining of specific RNA sequences, such as, for example, the synthesis of RNA probes or of oligoribonucleotides (Milligan et al.), or the expression of genes (see, in particular, Steen et al., Fuerst, et al. and Patent Applications WO 91/05,866 and EP 0,178,863), or alternatively gene amplification as described by Kievits, et al. or in Patent Applications WO 88/10,315 and WO 91/02,818, and U.S. Pat. No. 5,795,715, all of which are expressly incorporated herein by reference.

One of the distinctive features of most DNA-dependent RNA polymerases is that of initiating RNA synthesis according to a DNA template from a particular start site as a result of the recognition of a nucleic acid sequence, termed a promoter, which makes it possible to define the precise localization and the strand on which initiation is to be effected. Contrary to DNA-dependent DNA polymerases, polymerization by DNA-dependent RNA polymerases is not initiated from a 3′-OH end, and their natural substrate is an intact DNA double strand.

Compared to bacterial, eukaryotic or mitochondrial RNA polymerases, phage RNA polymerases are very simple enzymes. Among these, the best known are the RNA polymerases of bacteriophages T7, T3 and SP6. These enzymes are very similar to one another, and are composed of a single subunit of 98 to 100 kDa. Two other phage polymerases share these similarities: that of Klebsiella phage K11 and that of phage BA14 (Diaz et al.). Any DNA dependent RNA polymerase is expected to perform in conjunction with a functionally active promoter as desired in the present invention. These include, but are not limited to the above listed polymerases, active mutants thereof, E. coli RNA polymerase, and RNA polymerases I., II, and III from a variety of eukaryotic organisms.

Initiation of transcription with T7, SP6 RNA and T3 RNA Polymerases is highly specific for the T7, SP6 and T3 phage promoters, respectively. The properties and utility of these polymerases are well known to the art. Their properties and sources are described in U.S. Pat. Nos. (T7) 5,869,320; 4,952,496; 5,591,601; 6,114,152; (SP6) 5,026,645; (T3) 5,102,802; 5,891,681; 5,824,528; 5,037,745, all of which are expressly incorporated herein by reference.

Reaction conditions for use of these RNA polymerases are well known in the art, and are exemplified by those conditions provided in the examples and references. The result of contacting the appropriate template with an appropriate polymerase is the synthesis of an RNA product, which is typically single-stranded. Although under appropriate conditions, double stranded RNA may be made from a double stranded DNA template. See U.S. Pat. No. 5,795,715, incorporated herein by reference. The process of sequence specific synthesis may also be known as transcription, and the product the transcript, whether the product represents an entire, functional gene product or not.

dsRNA for use as siRNA may also be enzymatically synthesized through the use of RNA dependent RNA polymerases such as Q beta replicase, Tobacco mosaic virus replicase, brome mosaic virus replicase, potato virus replicase, etc. Reaction conditions for use of these RNA polymerases are well known in the art, and are exemplified by those conditions provided in the examples and references. Also see U.S. Pat. No. RE35,443, and U.S. Pat. No. 4,786,600, both of which are incorporated herein by reference. The result of contacting the appropriate template with an appropriate polymerase is the synthesis of an RNA product, which is typically double-stranded. Employing these RNA dependent RNA polymerases therefore may utilize a single stranded RNA or single stranded DNA template. If utilizing a single stranded DNA template, the enzymatic synthesis results in a hybrid RNA/DNA duplex that is also contemplated as useful as siRNA.

The templates for enzymatic synthesis of siRNA are nucleic acids, typically, though not exclusively DNA. A nucleic acid may be made by any technique known to one of ordinary skill in the art. Non-limiting examples of synthetic nucleic acid, particularly a synthetic oligonucleotide, include a nucleic acid made by in vitro chemical synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986, and U.S. Pat. No. 5,705,629, each incorporated herein by reference. A non-limiting example of enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of oligonucleotides described in U.S. Pat. No. 5,645,897, incorporated herein by reference. A non-limiting example of a biologically produced nucleic acid includes recombinant nucleic acid production in living cells (see for example, Sambrook, 2001, incorporated herein by reference).

The term “nucleic acid” will generally refer to at least one molecule or strand of DNA, RNA or a derivative or mimic thereof, comprising at least one nucleotide base, such as, for example, a naturally occurring purine or pyrimidine base found in DNA (e.g., adenine “A,” guanine “G,” thymine “T,” and cytosine “C”) or RNA (e.g. A, G, uracil “U,” and C). The term “nucleic acid” encompasses the terms “oligonucleotide” and “polynucleotide.” These definitions generally refer to at least one single-stranded molecule, but in specific embodiments will also encompass at least one additional strand that is partially, substantially or fully complementary to the at least one single-stranded molecule. Thus, a nucleic acid may encompass at least one double-stranded molecule or at least one triple-stranded molecule that comprises one or more complementary strand(s) or “complement(s)” of a particular sequence comprising a strand of the molecule.

As will be appreciated by one of skill in the art, the useful form of nucleotide or modified nucleotide to be incorporated will be dictated largely by the nature of the synthesis to be performed. Thus, for example, enzymatic synthesis typically utilizes the free form of nucleotides and nucleotide analogs, typically represented as nucleotide triphospates, or NTPs. These forms thus include, but are not limited to aminoallyl UTP, pseudo-UTP, 5-I-UTP, 5-I-CTP, 5-Br-UTP, alpha-S ATP, alpha-S CTP, alpha-S GTP, alpha-S UTP, 4-thio UTP, 2-thio-CTP, 2′NH₂ UTP, 2′NH₂ CTP, and 2′ F UTP. As will also be appreciated by one of skill in the art, the useful form of nucleotide for chemical syntheses may be typically represented as aminoallyl uridine, pseudo-uridine, 5-I-uridine, 5-I-cytidine, 5-Br-uridine, alpha-S adenosine, alpha-S cytidine, alpha-S guanosine, alpha-S uridine, 4-thio uridine, 2-thio-cytidine, 2′NH₂ uridine, 2′NH₂ cytidine, and 2′ F uridine. In the present invention, the listing of either form is non-limiting in that the choice of nucleotide form will be dictated by the nature of the synthesis to be performed. In the present invention, then, the inventors use the terms aminoallyl uridine, pseudo-uridine, 5-I-uridine, 5-I-cytidine, 5-Br-uridine, alpha-S adenosine, alpha-S cytidine, alpha-S guanosine, alpha-S uridine, 4-thio uridine, 2-thio-cytidine, 2′NH₂ uridine, 2′NH₂ cytidine, and 2′ F uridine generically to refer to the appropriate nucleotide or modified nucleotide, including the free phosphate (NTP) forms as well as all other useful forms of the nucleotides.

In certain embodiments, a “gene” refers to a nucleic acid that is transcribed. As used herein, a “gene segment” is a nucleic acid segment of a gene. In certain aspects, the gene includes regulatory sequences involved in transcription, or message production or composition. In particular embodiments, the gene comprises transcribed sequences that encode for a protein, polypeptide or peptide. In other particular aspects, the gene comprises a nucleic acid, and/or encodes a polypeptide or peptide-coding sequences of a gene that is defective or mutated in a hematopoietic and lympho-hematopoietic disorder. In keeping with the terminology described herein, an “isolated gene” may comprise transcribed nucleic acid(s), regulatory sequences, coding sequences, or the like, isolated substantially away from other such sequences, such as other naturally occurring genes, regulatory sequences, polypeptide or peptide encoding sequences, etc. In this respect, the term “gene” is used for simplicity to refer to a nucleic acid comprising a nucleotide sequence that is transcribed, and the complement thereof. In particular aspects, the transcribed nucleotide sequence comprises at least one functional protein, polypeptide and/or peptide encoding unit. As will be understood by those in the art, this functional term “gene” includes both genomic sequences, RNA or cDNA sequences, or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express using nucleic acid manipulation technology, proteins, polypeptides, domains, peptides, fusion proteins, mutants and/or such like. Thus, a “truncated gene” refers to a nucleic acid sequence that is missing a stretch of contiguous nucleic acid residues.

Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created:

n to n+y

where n is an integer from 1 to the last number of the sequence and y is the length of the nucleic acid segment minus one, where n+y does not exceed the last number of the sequence. Thus, for a 10-mer, the nucleic acid segments correspond to bases 1 to 10, 2 to 11, 3 to 12 . . . and/or so on. For a 15-mer, the nucleic acid segments correspond to bases 1 to 15, 2 to 16, 3 to 17 . . . and/or so on. For a 20-mer, the nucleic segments correspond to bases 1 to 20, 2 to 21, 3 to 22 . . . and/or so on.

The nucleic acid(s) of the present invention, regardless of the length of the sequence itself, may be combined with other nucleic acid sequences, including but not limited to, promoters, enhancers, polyadenylation signals, restriction enzyme sites, multiple cloning sites, coding segments, and the like, to create one or more nucleic acid construct(s). The overall length may vary considerably between nucleic acid constructs. Thus, a nucleic acid segment of almost any length may be employed, with the total length preferably being limited by the ease of preparation or use in the intended protocol.

To obtain the RNA corresponding to a given template sequence through the action of an RNA polymerase, it is necessary to place the target sequence under the control of the promoter recognized by the RNA polymerase.

The spacing between promoter elements frequently is flexible, so that promoter function is preserved when elements are inverted or moved relative to one another. The spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

T7, T3, or SP6 RNA polymerases display a high fidelity to their respective promoters. The natural promoters specific for the RNA polymerases of phages T7, T3 and SP6 are well known. Furthermore, consensus sequences of promoters are known to be functional as promoters for these polymerases. The bacteriophage promoters for T7, T3, and SP6 consist of 23 bp numbered −17 to +6, where +1 indicates the first base of the coded transcript. An important observation is that, of the +1 through +6 bases, only the base composition of +1 and +2 are critical and must be a G and purine, respectively, to yield an efficient transcription template. In addition, synthetic oligonucleotide templates only need to be double-stranded in the −17 to −1 region of the promoter, and the coding region can be all single-stranded. (See Milligan et al., 1987) This can reduce the cost of synthetic templates, since the coding region (i.e., from +1 on) can be left single-stranded and the short oligonucleotides required to render the promoter region double-stranded can be used with multiple templates. A further discussion of consensus promoters and a source of naturally occurring bacteriophage promoters is U.S. Pat. No. 5,891,681, specifically incorporated herein by reference.

Use of a T7, T3 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

When made in vitro, siRNA is formed from one or more strands of polymerized ribonucleotide. When formed of only one strand, it takes the form of a self-complementary hairpin-type or stem and loop structure that doubles back on itself to form a partial duplex. The self-duplexed portion of the RNA molecule may be referred to as the “stem” and the remaining, connecting single stranded portion referred to as the “loop” of the stem and loop structure. When made of two strands, they are substantially complementary.

It is contemplated that the region of complementarity in either case is at least 5 contiguous residues, though it is specifically contemplated that the region is at least or at most 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides. It is further understood that the length of complementarity between the dsRNA and the targeted mRNA may be any of the lengths identified above. Included within the term “dsRNA” is small interfering RNA (siRNA), which are generally 12-15 or 21-23 nucleotides in length and which possess the ability to mediate RNA interference. It is contemplated that RNase III dsRNA products of the invention may be 12, 13, 14, 15, 16, 17, 18, 19, 20, 21, 22, 23, 24, 25, 26, 27, 28, 29, 30 or more basepairs in length.

dsRNA capable of triggering RNAi has one region that is complementary to the targeted mRNA sequence and another region that is identical to the targeted mRNA sequence. Of course, it is understood that an mRNA is derived from genomic sequences or a gene. In this respect, the term “gene” is used for simplicity to refer to a functional protein, polypeptide, or peptide-encoding unit. As will be understood by those in the art, this functional term includes genomic sequences, cDNA sequences, and smaller engineered gene segments that express, or may be adapted to express, proteins, polypeptides, domains, peptides, fusion proteins, and mutants.

A dsRNA may be of the following lengths, or be at least or at most of the following lengths: 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs. It will be understood that these lengths refer either to a single strand of a two-stranded dsRNA molecule or to a single stranded dsRNA molecule having portions that form a double-stranded molecule.

Furthermore, outside regions of complementarity, there may be a non-complementarity region that is not complementary to another region in the other strand or elsewhere on a single strand. Non-complementarity regions may be at the 3′, 5′ or both ends of a complementarity region and they may number 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 5, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more bases.

The term “recombinant” may be used and this generally refers to a molecule that has been manipulated in vitro or that is the replicated or expressed product of such a molecule.

The term “nucleic acid” is well known in the art. A “nucleic acid” as used herein will generally refer to a molecule (one or more strands) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length. The use of “dsRNA” encompasses both “oligonucleotides” and “polynucleotides,” unless otherwise specified.

As used herein, “hybridization”, “hybridizes” or “capable of hybridizing” is understood to mean the forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions”, and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

1. Nucleic Acid Molecules

a. Nucleobases

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, caboxyalkyl, amino, hydroxyl, halogen (i.e., fluoro, chloro, bromo, or iodo), thiol or alkylthiol moeity. Preferred alkyl (e.g., alkyl, caboxyalkyl, etc.) moeities comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like. In the table below, non-limiting, purine and pyrimidine derivatives and analogs are also provided.

TABLE 3
Purine and Pyrmidine Derivatives or Analogs

| Abbr. | Modified base description |
|---|---|
| ac4c | 4-acetylcytidine |
| Chm5u | 5-(carboxyhydroxylmethyl) uridine |
| Cm | 2′-O-methylcytidine |
| Cmnm5s2u | 5-carboxymethylamino-methyl-2-thiouridine |
| Cmnm5u | 5-carboxymethylaminomethyluridine |
| D | Dihydrouridine |
| Fm | 2′-O-methylpseudouridine |
| Gal q | Beta,D-galactosylqueosine |
| Gm | 2′-O-methylguanosine |
| I | Inosine |
| I6a | N6-isopentenyladenosine |
| m1a | 1-methyladenosine |
| m1f | 1-methylpseudouridine |
| m1g | 1-methylguanosine |
| m1I | 1-methylinosine |
| m22g | 2,2-dimethylguanosine |
| m2a | 2-methyladenosine |
| m2g | 2-methylguanosine |
| m3c | 3-methylcytidine |
| m5c | 5-methylcytidine |
| m6a | N6-methyladenosine |
| m7g | 7-methylguanosine |
| Mam5u | 5-methylaminomethyluridine |
| Mam5s2u | 5-methoxyaminomethyl-2-thiouridine |
| Man q | Beta,D-mannosylqueosine |
| Mcm5s2u | 5-methoxycarbonylmethyl-2-thiouridine |
| Mcm5u | 5-methoxycarbonylmethyluridine |
| Mo5u | 5-methoxyuridine |
| Ms2i6a | 2-methylthio-N6-isopentenyladenosine |
| Ms2t6a | N-((9-beta-ribofuranosyl-2-methylthiopurine-6- |
| | yl)carbamoyl)threonine |
| Mt6a | N-((9-beta-ribofuranosylpurine-6-yl)N-methyl- |
| | carbamoyl)threonine |
| Mv | Uridine-5-oxyacetic acid methylester |
| o5u | Uridine-5-oxyacetic acid (v) |
| Osyw | Wybutoxosine |
| P | Pseudouridine |
| Q | Queosine |
| s2c | 2-thiocytidine |
| s2t | 5-methyl-2-thiouridine |
| s2u | 2-thiouridine |
| s4u | 4-thiouridine |
| T | 5-methyluridine |
| t6a | N-((9-beta-D-ribofuranosylpurine-6-yl)carbamoyl)threonine |
| Tm | 2′-O-methyl-5-methyluridine |
| Um | 2′-O-methyluridine |
| Yw | Wybutosine |
| X | 3-(3-amino-3-carboxypropyl)uridine, (acp3)u |

A nucleobase may be comprised in a nucleoside or nucleotide, using any chemical or natural synthesis method described herein or known to one of ordinary skill in the art. Such nucleobase may be labeled or it may be part of a molecule that is labeled and contains the nucleobase.

b. Nucleosides

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9 position of a purine or a 7-deazapurine to the 1′-position of a 5-carbon sugar. In another non-limiting example, a nucleoside comprising a pyrimidine nucleobase (i.e., C, T or U) typically covalently attaches a 1 position of a pyrimidine to a 1′-position of a 5-carbon sugar (Kornberg and Baker, 1992).

c. Nucleotides

As used herein, a “nucleotide” refers to a nucleoside further comprising a “backbone moiety.” A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. Other types of attachments are known in the art, particularly when a nucleotide comprises derivatives or analogs of a naturally occurring 5-carbon sugar or phosphorus moiety.

d. Nucleic Acid Analogs

A nucleic acid may comprise, or be composed entirely of, a derivative or analog of a nucleobase, a nucleobase linker moiety and/or backbone moiety that may be present in a naturally occurring nucleic acid. dsRNA with nucleic acid analogs may also be labeled according to methods of the invention. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, incorporated herein by reference).

Additional non-limiting examples of nucleosides, nucleotides or nucleic acids comprising 5-carbon sugar and/or backbone moiety derivatives or analogs, include those in: U.S. Pat. No. 5,681,947, which describes oligonucleotides comprising purine derivatives that form triple helixes with and/or prevent expression of dsDNA; U.S. Pat. Nos. 5,652,099 and 5,763,167, which describe nucleic acids incorporating fluorescent analogs of nucleosides found in DNA or RNA, particularly for use as fluorescent nucleic acids probes; U.S. Pat. No. 5,614,617, which describes oligonucleotide analogs with substitutions on pyrimidine rings that possess enhanced nuclease stability; U.S. Pat. Nos. 5,670,663, 5,872,232 and 5,859,221, which describe oligonucleotide analogs with modified 5-carbon sugars (i.e., modified 2′-deoxyfuranosyl moieties) used in nucleic acid detection; U.S. Pat. No. 5,446,137, which describes oligonucleotides comprising at least one 5-carbon sugar moiety substituted at the 4′ position with a substituent other than hydrogen that can be used in hybridization assays; U.S. Pat. No. 5,886,165, which describes oligonucleotides with both deoxyribonucleotides with 3′-5′ internucleotide linkages and ribonucleotides with 2′-5′ internucleotide linkages; U.S. Pat. No. 5,714,606, which describes a modified internucleotide linkage wherein a 3′-position oxygen of the internucleotide linkage is replaced by a carbon to enhance the nuclease resistance of nucleic acids; U.S. Pat. No. 5,672,697, which describes oligonucleotides containing one or more 5′ methylene phosphonate internucleotide linkages that enhance nuclease resistance; U.S. Pat. Nos. 5,466,786 and 5,792,847, which describe the linkage of a substituent moeity which may comprise a drug or label to the 2′ carbon of an oligonucleotide to provide enhanced nuclease stability and ability to deliver drugs or detection moieties; U.S. Pat. No. 5,223,618, which describes oligonucleotide analogs with a 2 or 3 carbon backbone linkage attaching the 4′ position and 3′ position of adjacent 5-carbon sugar moiety to enhanced cellular uptake, resistance to nucleases and hybridization to target RNA; U.S. Pat. No. 5,470,967, which describes oligonucleotides comprising at least one sulfamate or sulfamide internucleotide linkage that are useful as nucleic acid hybridization probe; U.S. Pat. Nos. 5,378,825, 5,777,092, 5,623,070, 5,610,289 and 5,602,240, which describe oligonucleotides with three or four atom linker moiety replacing phosphodiester backbone moiety used for improved nuclease resistance, cellular uptake and regulating RNA expression; U.S. Pat. No. 5,858,988, which describes hydrophobic carrier agent attached to the 2′-O position of oligonucleotides to enhanced their membrane permeability and stability; U.S. Pat. No. 5,214,136, which describes oligonucleotides conjugated to anthraquinone at the 5′ terminus that possess enhanced hybridization to DNA or RNA; enhanced stability to nucleases; U.S. Pat. No. 5,700,922, which describes PNA-DNA-PNA chimeras wherein the DNA comprises 2′-deoxy-erythro-pentofuranosyl nucleotides for enhanced nuclease resistance, binding affinity, and ability to activate RNase H; and U.S. Pat. No. 5,708,154, which describes RNA linked to a DNA to form a DNA-RNA hybrid; U.S. Pat. No. 5,728,525, which describes the labeling of nucleoside analogs with a universal fluorescent label.

Additional teachings for nucleoside analogs and nucleic acid analogs are U.S. Pat. No. 5,728,525, which describes nucleoside analogs that are end-labeled; U.S. Pat. Nos. 5,637,683, 6,251,666 (L-nucleotide substitutions), and 5,480,980 (7-deaza-2′ deoxyguanosine nucleotides and nucleic acid analogs thereof).

2. Preparation of Nucleic Acids

The present invention concerns various nucleic acids in different embodiments of the invention. There are a variety of ways to generate a dsRNA that can function as an siRNA or can be used as a substrate for a polypeptide with RNase III activity to generate siRNAs. In some embodiments, dsRNA is created by transcribing a DNA template. The DNA template may be comprised in a vector or it may be a non-vector template. Alternatively, a dsRNA may be created by hybridizing two synthetic, complementary RNA molecules or hybridizing a single synthetic RNA molecule with at least one complementarity region. Such nucleic acids may be made by any technique known to one of ordinary skill in the art, such as for example, chemical synthesis, enzymatic production or biological production.

a. Vectors

Nucleic acids of the invention, particularly DNA templates or DNA constructs for siRNA expression, may be produced recombinantly. Protein and polypeptides may be encoded by a nucleic acid molecule comprised in a vector. The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al., (2001) and Ausubel et al., 1994, both incorporated by reference. A vector may encode non-template sequences such as a tag or label. Useful vectors encoding such fusion proteins include pIN vectors (Inouye et al., 1985), vectors encoding a stretch of histidines, and pGEX vectors, for use in generating glutathione S-transferase (GST) soluble fusion proteins for later purification and separation or cleavage.

A DNA construct refers to a plasmid, viral DNA, or linear DNA molecule bearing an siRNA sequence that is expressed by an adjacent or otherwise upstream RNA polymerase promoter element. Thus far, the expression of siRNAs from DNA constructs has primarily been via RNA polymerase III (Brummelkamp et al 2002 and Paddison et al. 2002), though a recent publication describes the expression of functional siRNAs from an RNA Polymerase II promoter (Xia et al 2002). SiRNA cocktails can be generated in mammalian cells if one or more DNA constructs bearing one or more siRNA expression domains are transfected or transduced into cells.

The term “expression vector” or “expression construct” refers to a vector or construct containing a nucleic acid sequence coding for at least part of a gene product capable of being transcribed. In some cases, RNA molecules are then translated into a protein, polypeptide, or peptide. In other cases, these sequences are not translated, for example, in the production of siRNAs, antisense molecules, or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

The term “expression domain” refers to parts of an expression construct that include a promoter element operatively linked to a nucleic acid sequence coding for all or at least part of a gene product or siRNA. As used herein, an expression construct may contain 1, 2, 3, 4, 5, 6, 7, 8, 9, 10 or more expression domains each of which may or may not be independently transcribed. An expression construct containing multiple expression domains may contain 1, 2, 3, 4, 5, 6, 7, 8, 9, 10 or more of the same or 1, 2, 3, 4, 5, 6, 7, 8, 9, 10 or more different siRNAs and combinations thereof.

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter (examples include the bacterial promoters SP6, T3, and T7), which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202, U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it may be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein or RNA expression, for example, see Sambrook et al. (2001), incorporated herein by reference. The promoters employed may be constitutive, tissue-specific, inducible, and/or useful under the appropriate conditions to direct high level expression from the introduced DNA segment. The promoter may be heterologous or endogenous.

Other elements of a vector are well known to those of skill in the art. A vector may include a polyadenylation signal, an initiation signal, an internal ribosomal binding site, a multiple cloning site, a selective or screening marker, a termination signal, a splice site, an origin of replication, or a combination thereof.

b. In Vitro Synthesis of dsRNA

A DNA template may be used to generate complementary RNA molecule(s) to generate a double-stranded RNA molecule that can be a functional siRNA or a substrate for RNase III. One or two DNA templates may be employed to generate a dsRNA. In some embodiments, the DNA template can be part of a vector or plasmid, as described herein. Alternatively, the DNA template for RNA may be created by an amplification method.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred. Pairs of primers designed to selectively hybridize to nucleic acids corresponding to the target gene are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids contain one or more mismatches with the primer sequences. Once hybridized, the template-primer complex is contacted with one or more enzymes that facilitate template-dependent nucleic acid synthesis. Multiple rounds of amplification are conducted until a sufficient amount of product is produced.

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety. A reverse transcriptase PCR™ amplification procedure may be performed to quantify the amount of mRNA amplified. Methods of reverse transcribing RNA into cDNA are well known (see Sambrook et al., 2001). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety. Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase copies the replicative sequence which may then be detected. An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; PCT Application WO 88/10315, incorporated herein by reference in their entirety). EP Application 329 822 disclose a nucleic acid amplification process involving cyclically synthesizing ssRNA, ssDNA, and dsDNA, which may be used in accordance with the present invention. PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989).

c. Chemical Synthesis

Nucleic acid synthesis is performed according to standard methods. See, for example, Itakura and Riggs (1980). Additionally, U.S. Pat. No. 4,704,362, U.S. Pat. No. 5,221,619, and U.S. Pat. No. 5,583,013 each describe various methods of preparing synthetic nucleic acids. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 2001, incorporated herein by reference).

Oligonucleotide synthesis is well known to those of skill in the art. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

Basically, chemical synthesis can be achieved by the diester method, the triester method polynucleotides phosphorylase method and by solid-phase chemistry. These methods are discussed in further detail below.

Diester method. The diester method was the first to be developed to a usable state, primarily by Khorana and co-workers. (Khorana, 1979). The basic step is the joining of two suitably protected deoxynucleotides to form a dideoxynucleotide containing a phosphodiester bond. The diester method is well established and has been used to synthesize DNA molecules (Khorana, 1979).

Triester method. The main difference between the diester and triester methods is the presence in the latter of an extra protecting group on the phosphate atoms of the reactants and products (Itakura et al., 1975). The phosphate protecting group is usually a chlorophenyl group, which renders the nucleotides and polynucleotide intermediates soluble in organic solvents. Therefore purification's are done in chloroform solutions. Other improvements in the method include (i) the block coupling of trimers and larger oligomers, (ii) the extensive use of high-performance liquid chromatography for the purification of both intermediate and final products, and (iii) solid-phase synthesis.

Polynucleotide phosphorylase method. This is an enzymatic method of DNA synthesis that can be used to synthesize many useful oligonucleotides (Gillam et al., 1978; Gillam et al., 1979). Under controlled conditions, polynucleotide phosphorylase adds predominantly a single nucleotide to a short oligonucleotide. Chromatographic purification allows the desired single adduct to be obtained. At least a trimer is required to start the procedure, and this primer must be obtained by some other method. The polynucleotide phosphorylase method works and has the advantage that the procedures involved are familiar to most biochemists.

Solid-phase methods. Drawing on the technology developed for the solid-phase synthesis of polypeptides, it has been possible to attach the initial nucleotide to solid support material and proceed with the stepwise addition of nucleotides. All mixing and washing steps are simplified, and the procedure becomes amenable to automation. These syntheses are now routinely carried out using automatic nucleic acid synthesizers.

Phosphoramidite chemistry (Beaucage and Lyer, 1992) has become by far the most widely used coupling chemistry for the synthesis of oligonucleotides. As is well known to those skilled in the art, phosphoramidite synthesis of oligonucleotides involves activation of nucleoside phosphoramidite monomer precursors by reaction with an activating agent to form activated intermediates, followed by sequential addition of the activated intermediates to the growing oligonucleotide chain (generally anchored at one end to a suitable solid support) to form the oligonucleotide product.

3. Nucleic Acid Purification

A nucleic acid may be purified on polyacrylamide gels, cesium chloride centrifugation gradients, or by any other means known to one of ordinary skill in the art (see for example, Sambrook (2001), incorporated herein by reference). Alternatively, a column, filter, or cartridge containing an agent that binds to the nucleic acid, such as a glass fiber, may be employed.

Following any amplification or transcription reaction, it may be desirable to separate the amplification or transcription product from the template and/or the excess primer. In one embodiment, products are separated by agarose, agarose-acrylamide or polyacrylamide gel electrophoresis using standard methods (Sambrook et al., 2001). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 2001). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

4. Nucleic Acid Transfer

Suitable methods for nucleic acid delivery to effect RNAi according to the present invention are believed to include virtually any method by which a nucleic acid (e.g., DNA, RNA, including viral and nonviral vectors) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

There are a number of ways in which expression vectors may be introduced into cells to generate dsRNA. In certain embodiments of the invention, the expression vector comprises a virus or engineered vector derived from a viral genome, while in other embodiments, it is a nonviral vector. Other expression systems are also readily available.

5. Host Cells and Target Cells

The cell containing the target gene may be derived from or contained in any organism (e.g., plant, animal, protozoan, virus, bacterium, or fungus). The plant may be a monocot, dicot or gynmosperm; the animal may be a vertebrate or invertebrate. Preferred microbes are those used in agriculture or by industry, and those that a pathogenic for plants or animals. Fungi include organisms in both the mold and yeast morphologies. Examples of vertebrates include fish and mammals, including cattle, goat, pig, sheep, hamster, mouse, rate and human; invertebrate animals include nematodes, insects, arachnids, and other arthropods. Preferably, the cell is a vertebrate cell. More preferably, the cell is a mammalian cell.

The cell having the target gene may be from the germ line or somatic, totipotent or pluripotent, dividing or non-dividing, parenchyma or epithelium, immortalized or transformed, or the like. The cell can be a gamete or an embryo; if an embryo, it can be a single cell embryo or a constituent cell or cells from a multicellular embryo. The term “embryo” thus encompasses fetal tissue. The cell having the target gene may be an undifferentiated cell, such as a stem cell, or a differentiated cell, such as from a cell of an organ or tissue, including fetal tissue, or any other cell present in an organism. Cell types that are differentiated include adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes, and cells, of the endocrine or exocrine glands.

As used herein, the terms “cell,” “cell line,” and “cell culture” may be used interchangeably. All of these terms also include their progeny, which is any and all subsequent generations formed by cell division. It is understood that all progeny may not be identical due to deliberate or inadvertent mutations. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a small, interfering RNA or a template construct encoding such an RNA has been introduced. Therefore, recombinant cells are distinguishable from naturally occurring cells which do not contain a recombinantly introduced nucleic acid.

In certain embodiments, it is contemplated that RNAs or proteinaceous sequences may be co-expressed with other selected RNAs or proteinaceous sequences in the same host cell. Co-expression may be achieved by co-transfecting the host cell with two or more distinct recombinant vectors. Alternatively, a single recombinant vector may be constructed to include multiple distinct coding regions for RNAs, which could then be expressed in host cells transfected with the single vector.

A tissue may comprise a host cell or cells to be transformed or contacted with a nucleic acid delivery composition and/or an additional agent. The tissue may be part or separated from an organism. In certain embodiments, a tissue and its constituent cells may comprise, but is not limited to, blood (e.g., hematopoietic cells (such as human hematopoietic progenitor cells, human hematopoietic stem cells, CD34⁺ cells CD4⁺ cells), lymphocytes and other blood lineage cells), bone marrow, brain, stem cells, blood vessel, liver, lung, bone, breast, cartilage, cervix, colon, cornea, embryonic, endometrium, endothelial, epithelial, esophagus, facia, fibroblast, follicular, ganglion cells, glial cells, goblet cells, kidney, lymph node, muscle, neuron, ovaries, pancreas, peripheral blood, prostate, skin, skin, small intestine, spleen, stomach, testes.

In certain embodiments, the host cell or tissue may be comprised in at least one organism. In certain embodiments, the organism may be, human, primate or murine. In other embodiments the organism may be any eukaryote or even a prokayrote (e.g., a eubacteria, an archaea), as would be understood by one of ordinary skill in the art (see, for example, webpage http://phylogeny.arizona.edu/tree/phylogeny.html). One of skill in the art would further understand the conditions under which to incubate all of the above described host cells to maintain them and to permit their division to form progeny.

6. Labels and Tags

dsRNA may be labeled with a radioactive, enzymatic, colorimetric, or other label or tag for detection or isolation purposes. Nucleic acids may be labeled with fluorescence in some embodiments of the invention. The fluorescent labels contemplated for use as conjugates include, but are not limited to, Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, TET, Tetramethylrhodamine, and/or Texas Red. For exemplary methods and compositions for labeling RNA, dsRNA, or siRNA see U.S. Provisional Application Ser. No. 60/388,547 or U.S. patent application Ser. No. 10/029,397, each of which is hereby incorporated by reference.

It is contemplated that dsRNA may be labeled with two different labels. Furthermore, fluorescence resonance energy transfer (FRET) may be employed in methods of the invention (e.g., Klostermeier et al., 2002; Emptage, 2001; Didenko, 2001, each incorporated by reference).

A number of techniques for visualizing or detecting labeled dsRNA are readily available. The reference by Stanley T. Crooke, 2000 has a discussion of such techniques (Chapter 6) which is incorporated by reference. Such techniques include, microscopy, arrays, Fluorometry, Light cyclers or other real time PCR machines, FACS analysis, scintillation counters, Phosphoimagers, Geiger counters, MRI, CAT, antibody-based detection methods (Westerns, immunofluorescence, immunohistochemistry), histochemical techniques, HPLC (Griffey et al., 1997, spectroscopy, capillary gel electrophoresis (Cummins et al., 1996), spectroscopy; mass spectroscopy; radiological techniques; and mass balance techniques. Alternatively, nucleic acids may be labeled or tagged to allow for their efficient isolation. In other embodiments of the invention, nucleic acids are biotinylated.

7. Libraries and Arrays

The present methods and kits may be employed for high volume screening. A library of candidate siRNA cocktails, siRNA cocktails or DNA constructs expressing siRNA cocktails can be created using methods of the invention. This library may then be used in high throughput assays, including microarrays. Specifically contemplated by the present inventors are chip-based nucleic acid technologies such as those described by Sabatini (2001) Briefly, nucleic acids can be immobilized on solid supports. Cells can then be overlaid on the solid support and take up the nucleic acids at the defined locations. The impact on the cells can then be measured to identify cocktails that are having a desirable effect.

### III. Kits

Any of the compositions described herein may be comprised in a kit. In a non-limiting example, reagents for generating or assembling siRNA cocktails or candidate siRNA molecules are included in a kit. The kit may further include individual siRNAs that can be mixed to create an siRNA cocktail or individual DNA constructs that can be mixed and transfected or transduced into cells wherein they express a cocktail of siRNAs. The kit may also include multiple DNA templates encoding siRNAs to multiple sites on one or more genes that when transcribed create an siRNA cocktail. The kit may also comprise reagents for creating or synthesizing the dsRNA and a polypeptide with RNAse III activity that can be used in combination to create siRNA cocktails. It may also include one or more buffers, such as a nuclease buffer, transcription buffer, or a hybridization buffer, compounds for preparing the DNA template or the dsRNA, and components for isolating the resultant template, dsRNA, or siRNA. Other kits of the invention may include components for making a nucleic acid transfection array comprising siRNA cocktails or DNA constructs capable of expressing siRNA cocktails, and thus, may include, for example, a solid support.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the nucleic acids, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means. In some embodiments, labeling dyes are provided as a dried power. It is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 μg or at least or at most those amounts of dried dye are provided in kits of the invention. The dye may then be resuspended in any suitable solvent, such as DMSO.

The container means will generally include at least one vial, test tube, flask, bottle, syringe and/or other container means, into which the nucleic acid formulations are placed, preferably, suitably allocated. The kits may also comprise a second container means for containing a sterile, pharmaceutically acceptable buffer and/or other diluent.

The kits of the present invention will also typically include a means for containing the vials in close confinement for commercial sale, such as, e.g., injection and/or blow-molded plastic containers into which the desired vials are retained.

Such kits may also include components that facilitate isolation of the DNA construct, DNA template, long dsRNA, or siRNA. It may also include components that preserve or maintain the nucleic acids or that protect against their degradation. Such components may be RNAse-free or protect against RNAses, such as RNase inhibitors. Such kits generally will comprise, in suitable means, distinct containers for each individual reagent or solution.

A kit will also include instructions for employing the kit components as well the use of any other reagent not included in the kit. Instructions may include variations that can be implemented.

Kits of the invention may also include one or more of the following in addition to a polypeptide with RNase III activity: 1) RNase III buffer; 2) Control dsRNA, including but not limited to, GAPDH siRNA or c-myc siRNA (shown in Examples); 3) SP6, T3, and/or T7 polymerase; 4) SP6, T3, and/or T7 polymerase buffer; 5) dNTPs and/or NTPs; 6) nuclease-free water; 7) RNase-free containers, such as 1.5 ml tubes; 8) RNase-free elution tubes; 9) glycogen; 10) ethanol; 11) sodium acetate; 12) ammonium acetate; 13) agarose or acrylamide gel; 14) nucleic acid size marker; 15) RNase-free tube tips; or 16) RNase or DNase inhibitors.

It is contemplated that such reagents are embodiments of kits of the invention. Such kits, however, are not limited to the particular items identified above and may include any labeling reagent or reagent that promotes or facilitates the labeling of a nucleic acid to trigger RNAi.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Co-Transfection of siRNAs Designed for Target Sites of GAPDH

Four siRNAs specific to GAPDH were designed. These siRNAs were prepared by in vitro transcription using the following procedure: The following synthetic DNA oligomers were purchased from Integrated DNA Technologies (Table 4):

In separate reactions, the T7 promoter primer was mixed with each of the sense and antisense templates in separate reactions and converted to transcription templates. Templates for in vitro transcription must be double-stranded over the length of the promoter sequence (Milligan et al. 1987). Making the entire template double-stranded improves the transcription of siRNAs, therefore the following procedure is used to convert DNA oligonucleotides to transcription templates for siRNA synthesis.

TABLE 4

| Name | DNA Sequence (5′ to 3′) | SEQ ID NO: |
|---|---|---|
| T7 Promoter Primer: | GGTAATACGACTCACTATAGGGAGACAGG | SEQ ID NO: 7 |
| 5′ GAPDH sense: | AAGTGGATATTGTTGCCATCACCTGTCTC | SEQ ID NO: 8 |
| 5′ GAPDH antisense: | AATGATGGCAACAATATCCACCCTGTCTC | SEQ ID NO: 9 |
| 5′ Medial GAPDH | AAGGTCATCCATGACAACTCCTGTCTC | SEQ ID NO: 10 |
| sense | | |
| 5′ Medial GAPDH | AAAAAGTTGTCATGGATGACCCCTGTCTC | SEQ ID NO: 11 |
| antisense | | |
| 3′ Medial GAPDH | AAGCTTCACTGGCATGGCCTTCCCTGTCTC | SEQ ID NO: 12 |
| sense | | |
| 3′ Medial GAPDH | AAGAAGGCCATGCCAGTGAGCCCTGTCTC | SEQ ID NO: 13 |
| antisense | | |
| 3′ GAPDH sense | AACAGGGTGGTGGACCTCATGCCTGTCTC | SEQ ID NO: 14 |
| 3′ GAPDH antisense | AACATGAGGTCCACCACCCTGCCTGTCTC | SEQ ID NO: 15 |

The DNA templates were diluted to 100 μm in nuclease-free water. Two μl of each DNA template was mixed with 2 μl of 100 μM Promoter Primer and 6 μl of Hybridization Buffer (20 mM Tris pH 7.0, 100 mM KCl, 1 mM EDTA). The oligonucleotide mixtures were heated to 70° C. for five minutes, then incubate at 37° C. for five minutes. Two μl of 10× reaction Buffer (150 mM Tris pH 7.0, 850 mM KCl, 50 mM MgCl₂, 50 mM (NH₄)₂SO₄), 2 μl of 10 dNTP mix (2.5 mM dATP, 2.5 mM dCTP, 2.5 mM dGTP, and 2.5 mM dTTP), 4 μl of water, and 2 μl of 5 U/ml klenow DNA polymerase was added to each oligonucleotide mixture. The reaction was incubated at 37° C. for thirty minutes.

The templates were transcribed using T7 RNA polymerase by mixing 2 μl siRNA DNA Template; 2 μl 75 mM ATP; 2 μl 75 mM CTP; 2 μl 75 mM GTP; 2 μl 75 mM UTP; 2 μl 10× Transcription Buffer (400 mM Tris pH 8.0, 240 mM MgCl₂, 20 mM Spermidine, 100 mM DTT); 6 μl Nuclease-Free Water; and 2 μl T7 RNA Polymerase (T7 RNA Polymerase—200 U/μl, inorganic Pyrophosphatase (IPP) 0.05 U/μl, RNase Inhibitor 0.3 U/μl, superasin 2 U/μl, 1% chaps)

This reaction mix was incubated for two to four hours at 37° C. The RNA products were then mixed and incubated overnight at 37° C. to facilitate annealing of the complementary strands of the siRNAs. The leader sequences were removed by treatment with RNase T1 and the resulting siRNAs were gel purified.

10× Transcription Buffer (400 mM Tris pH 8.0, 240 mM MgCl₂, 20 mM Spermidine, 100 mM T7 RNA Polymerase (T7 RNA Polymerase-200 U/μl, Inorganic Pyrophosphatase (IPP) 0.05 U/μl, RNase Inhibitor 0.3 U/μl, Superasin 2 U/μl, 1% chaps). HeLa cells were transfected with 10 nM of each of the GAPDH-specific siRNAs using the protocol presented in above. Forty-eight hours after transfection, the cells were harvested and RNA was isolated using the RNAquesous kit (Ambion). Equal amounts of the RNA samples were fractionated by agarose gel electrophoresis and transferred to positively charged nylon membranes using the NorthernMax-Gly kit (Ambion). The Northern blots were probed for GAPDH, cyclophilin, and 28S rRNA using the reagents and protocols of the NorthernMax-Gly kit. The Northern blots were exposed to film. Two of the siRNAs provide reasonable reductions in GAPDH mRNA and the pool of the four siRNAs provides the greatest levels of knockdown.

### Example 2

### Real-Time PCR Analysis of Multiple siRNAs on the Rho, CDC 2, and Survivin Genes

Pools of four different siRNAs were prepared for each of Rho, CDC 2, and Survivin genes using the siRNA transcription procedure described above, see Example 6. Each siRNA was prepared for transfection and mixed with cells at a final concentration of 10 nM. In a fifth transfection, all four siRNAs at a final concentration of 10 nM were mixed with the same cells. Forty-eight hours after transfection, RNA was isolated from the mammalian cells using the RNAqueous-4-PCR kit (Ambion). 0.5 pg of the RNA samples were reverse transcribed using the RetroScript kit with random primers (Ambion). Equal amounts of cDNA were applied to real-time PCR assays using SYBR green detection (Molecular Probes). The level of target gene expression was measured as a function of the difference in Ct values between cells transfected with the target-specific siRNAs and cell transfected with a negative control siRNA. The Ct values from each sample were normalized using the Ct values derived from the amplification of GAPDH in the same cDNA samples.

TABLE 5

| SiRNA | Reduction in Target mRNA expression |
|---|---|
| Rho 1 | 83% |
| Rho 2 | <50% |
| Rho 3 | <50% |
| Rho 4 | <50% |
| Rho Cocktail | 93% |
| CDC2 1 | 50% |
| CDC 2 2 | 69% |
| CDC 2 3 | <50% |
| CDC 2 4 | <50% |
| CDC 2 cocktail | 96% |
| Survivin 1 | <50% |
| Survivin 2 | 70% |
| Survivin 3 | 50% |
| Survivin 4 | <50% |
| Survivin cocktail | 89% |

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references are specifically incorporated herein by reference.
U.S. Pat. No. RE 35,443
U.S. Prov. application Ser. No. 10/029,397
U.S. Prov. Appl. 60/353,332
U.S. Prov. Appl. 60/388,547
U.S. Prov. Appl. 60/402,347
U.S. Pat. No. 4,554,101
U.S. Pat. No. 4,659,774
U.S. Pat. No. 4,682,195
U.S. Pat. No. 4,683,202
U.S. Pat. No. 4,684,611
U.S. Pat. No. 4,704,362
U.S. Pat. No. 4,786,600
U.S. Pat. No. 4,800,159
U.S. Pat. No. 4,816,571
U.S. Pat. No. 4,883,750
U.S. Pat. No. 4,952,496
U.S. Pat. No. 4,952,500
U.S. Pat. No. 4,959,463
U.S. Pat. No. 5,026,645
U.S. Pat. No. 5,037,745
U.S. Pat. No. 5,102,802
U.S. Pat. No. 5,141,813
U.S. Pat. No. 5,214,136
U.S. Pat. No. 5,221,619
U.S. Pat. No. 5,223,618
U.S. Pat. No. 5,264,566
U.S. Pat. No. 5,279,721
U.S. Pat. No. 5,302,523
U.S. Pat. No. 5,322,783
U.S. Pat. No. 5,378,825
U.S. Pat. No. 5,384,253
U.S. Pat. No. 5,428,148
U.S. Pat. No. 5,446,137
U.S. Pat. No. 5,464,765
U.S. Pat. No. 5,466,786
U.S. Pat. No. 5,470,967
U.S. Pat. No. 5,480,980
U.S. Pat. No. 5,538,877
U.S. Pat. No. 5,538,880
U.S. Pat. No. 5,550,318
U.S. Pat. No. 5,554,744
U.S. Pat. No. 5,563,055
U.S. Pat. No. 5,574,146
U.S. Pat. No. 5,580,859
U.S. Pat. No. 5,583,013
U.S. Pat. No. 5,589,466
U.S. Pat. No. 5,591,601
U.S. Pat. No. 5,591,616
U.S. Pat. No. 5,602,240
U.S. Pat. No. 5,602,244
U.S. Pat. No. 5,610,042
U.S. Pat. No. 5,610,289
U.S. Pat. No. 5,614,617
U.S. Pat. No. 5,623,070
U.S. Pat. No. 5,637,683
U.S. Pat. No. 5,645,897
U.S. Pat. No. 5,652,099
U.S. Pat. No. 5,656,610
U.S. Pat. No. 5,670,663
U.S. Pat. No. 5,672,697
U.S. Pat. No. 5,681,947
U.S. Pat. No. 5,700,922
U.S. Pat. No. 5,702,932
U.S. Pat. No. 5,705,629
U.S. Pat. No. 5,708,154
U.S. Pat. No. 5,714,606
U.S. Pat. No. 5,728,525
U.S. Pat. No. 5,736,524
U.S. Pat. No. 5,763,167
U.S. Pat. No. 5,777,092
U.S. Pat. No. 5,780,448
U.S. Pat. No. 5,789,215
U.S. Pat. No. 5,792,847
U.S. Pat. No. 5,795,715
U.S. Pat. No. 5,824,528
U.S. Pat. No. 5,840,873
U.S. Pat. No. 5,843,640
U.S. Pat. No. 5,843,650
U.S. Pat. No. 5,843,651
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,849,487
U.S. Pat. No. 5,849,497
U.S. Pat. No. 5,849,546
U.S. Pat. No. 5,849,547
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,858,988
U.S. Pat. No. 5,859,221
U.S. Pat. No. 5,861,244
U.S. Pat. No. 5,863,732
U.S. Pat. No. 5,863,753
U.S. Pat. No. 5,866,331
U.S. Pat. No. 5,866,366
U.S. Pat. No. 5,869,320
U.S. Pat. No. 5,872,232
U.S. Pat. No. 5,882,864
U.S. Pat. No. 5,886,165
U.S. Pat. No. 5,891,681
U.S. Pat. No. 5,905,024
U.S. Pat. No. 5,910,407
U.S. Pat. No. 5,912,124
U.S. Pat. No. 5,912,145
U.S. Pat. No. 5,912,148
U.S. Pat. No. 5,916,776
U.S. Pat. No. 5,916,779
U.S. Pat. No. 5,919,630
U.S. Pat. No. 5,922,574
U.S. Pat. No. 5,925,517
U.S. Pat. No. 5,928,862
U.S. Pat. No. 5,928,869
U.S. Pat. No. 5,928,905
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,825
U.S. Pat. No. 5,939,291
U.S. Pat. No. 5,942,391
U.S. Pat. No. 5,945,100
U.S. Pat. No. 5,981,274
U.S. Pat. No. 5,994,624
U.S. Pat. No. 6,114,152
U.S. Pat. No. 6,251,666
PCT Applic. PCT/US87/00880
PCT Applic. PCT/US89/01025
PCT Applic. WO 00/44914
PCT Applic. WO 01/68836
PCT Applic. WO 01/36646
PCT Applic. WO 89/06700
PCT Applic. WO 99/32619
PCT Applic. WO 88/10315
PCT Applic. WO 94/09699
PCT Applic. WO 90/07641
PCT Applic. WO 95/06128
PCT Applic. WO 91/05,866
PCT Applic. WO 91/02,818
GB Application 2 202 328
European Appl. 0,178,863
European Appl. 266,032
European Appl. 320,308
European Appl. 329,822
Antisense Drug Technology, Stanley T. Crooke (ed), Marcel Dekker and Co, Basel, Switzerland, Chapter 6, 2001.
Ausubel et al., In: Current Protocols in Molecular Biology, John, Wiley and Sons, Inc, New York, 1994.
Beaucage, and Lyer, Tetrahedron, 48:2223-2311, 1992.
Bernstein et al., Nature, 409: 363-366, 2001.
Biocomputing: Informatics and Genome Projects, Smith (Ed.), Academic Press, NY, 1993.
Brummelkamp et al., Science, 296(5567):550-553, 2002
Caplen et al., Proc Natl Acad Sci USA, 98: 9742-9747, 2001.
Chen and Okayama, Mol. Cell. Biol., 7(8):2745-2752, 1987.
Cogoni and Macino, Science, 286:342-2344, 1999.
Cogoni. and Macino, Nature, 399:166-169, 1999.
Computational Molecular Biology, Oxford University Press, Lesk (Ed.), NY, 1988.
Cummins et al., In IRT: Nucleosides and nucleosides, La Jolla Calif., 72, 1996.
Dalmay et al. EMBO J., 20:2069-2078, 2001.
Dalmay et al., Cell, 101:543-553, 2000.
Devereux et al., Nucl. Acids Res., 12(Pt1):387-395, 1984.
Diaz and Ortega, Nucl. Acids Res., 12(13):5175-5191, 1984.
Didenko, Biotechniques, 31(5):1106-16, 1118, 1120-1, 2001.
Elbashir et al., Nature, 411:494-498, 2001.
Emptage et al.: Neuron, 2001 January; 29(1):197-208, 2001.
Fechheimer et al., Proc. Natl. Acad. Sci. USA, 84:8463-8467, 1987.
Fire et al., Nature, 391:806-811, 1998.
Fraley et al., Proc. Natl. Acad. Sci. USA, 76:3348-3352, 1979.
Froehler et al., Nucleic Acids Res., 14(13):5399-5407, 1986.
Frohman, In: PCR Protocols: A Guide To Methods and Applications, Academic Press, NY, 1990.
Fuerst et al., Mol. Cell. Biol., 7(7):2538-2544, 1987.
Gillam et al., J. Biol. Chem., 253:2532, 1978.
Gillam et al., Nucleic Acids Res., 6:2973, 1979.
Gopal, Mol. Cell. Biol., 5:1188-1190, 1985.
Graham and Van Der Eb, Virology, 52:456-467, 1973.
Griffey et al., J. Mass. Spectrom., 32(3):305-13, 1997.
Grishok et al., Cell, 106: 23-34, 2001.
Hamilton and Baulcombe, Science, 286:950-952, 1999.
Hammond et al., Nat. Rev. Genet., 2(2):110-9, 2001.
Harland and Weintraub, J. Cell. Biol., 101:1094-1099, 1985.
Higgins et al., Comput. Appl. Biosci., 8(2):189-191, 1992.
Holen et al. Nucl. Acids Res. 30(8):1757-1766, 2002.
Hutvagner et al., Science, 293:834-838, 2001.
Innis et al., Proc. Natl. Acad. Sci. USA, 85(24):9436-9440, 1988.
Inouye and Inouye, Nucleic Acids Res., 13:3101-3109, 1985.
Itakura and Riggs, Science, 209:1401-1405, 1980.
Itakura et al., J. Biol. Chem., 250:4592 1975.
Jacque et al., Nature, 2002.
Johnson et al., In: Biotechnology and Pharmacy, Pezzuto et al., (eds), Chapman and Hall, New York, 1993.
Kaeppler et al., Plant Cell Reports, 9:415-418, 1990.
Kaneda et al., Science, 243:375-378, 1989.
Kato et al, J. Biol. Chem., 266:3361-3364, 1991.
Ketting et al., Cell, 99:133-141, 1999.
Khorana, Science, 203, 614 1979.
Kievits et al.: J. Virol. Methods, 35(3):273-286, 1991.
Klostermeier and Millar, Biopolymers, 61(3):159-79, 2001-2002
Knight et al., Science, 2:2, 2001.
Kornberg and Baker, In: DNA Replication, 2nd Ed., Freeman, San Francisco, 1992.
Kwoh et al., Proc. Natl. Acad. Sci. USA, 86: 1173, 1989.
Kyte and Doolittle, J. Mol. Biol., 157(1):105-132, 1982.
Lee et al., DNA Cell Biol., 16(11):1267-1275, 1997.
Lin and Avery, Nature, 402:128-129, 1999.
Martinez et al., Cell, 110(5):563, 2002.
Milligan et al., J. Chromatogr., 411:481-485, 1987.
Miyagishi and Taira, Biotechnol., 5:497-500, 2002.
Montgomery et al., Proc. Natl. Acad. Sci. USA, 95:155-2-15507, 1998.
Mourrain et al., Cell, 101:533, 2000.
Nicolau and Sene, Biochim. Biophys. Acta, 721:185-190, 1982.
Nicolau et al., Methods Enzymol., 149:157-176, 1987.
Novina et al., Nat. Med., 8:681-686, 2002.
Ohara et al., Proc. Natl. Acad. Sci. USA, 86:5673-5677, 1989.
Omirulleh et al., Plant Mol. Biol., 21(3):415-428, 1993.
Paddison et al. Genes and Development 16:948-958, 2002.
Paul et al., Nat. Biotechnol., 20:505-508, 2002.
Potrykus et al., Mol. Gen. Genet., 199(2):169-77, 1985.
Rippe et al., Mol. Cell. Biol., 10:689-695, 1990.
Sabatini, Am. J. Med. Sci., 322(4):175-178, 2001.
Sambrook et al., In: Molecular cloning, Cold Spring Harbor Laboratory Press, Cold Spring Harbor, N.Y., 2001.
Scheit, In: Synthesis and Biological Function, Wiley-Interscience, NY, 171-172, 1980.
Sharp and Zamore, Science, 287:2431-2433, 2000.
Sharp, Genes Dev., 13:139-141, 1999.
Smardon et al., Curr. Biol., 10:169-178, 2000.
Steen et al., Prog. Nucl. Acid Res. Mol. Biol., 33:1-18, 1986.
Sui et al., Proc. Natl. Acad. Sci. USA, 99(8):5515-5520, 2002.
Tabara et al., Cell, 99:123-132, 1999.
Thompson et al., Nucleic Acids Res., 22(22):4673-4680, 1994.
Tuschl, Chembiochem., 2:239-245, 2001.
Walker et al., Proc. Natl. Acad. Sci. USA, 89:392-396 1992.
Waterhouse et al., Nature, 411:834-842, 2001.
Wong et al., Gene, 10:87-94, 1980.
Wu-Scharf et al., Science, 290:1159-1162, 2000.
Xia et al. Nature Biotechnology 20(10):1006-10, 2002
Yu et al., Proc. Natl. Acad. Sci. USA, 99:6047-6052, 2002.
Zamore et al., Cell, 101:25-33, 2000.
Zamore, Nat. Struct. Biol., 8:746-750, 2001.

1.-56. (canceled)

57. A composition for reducing the expression of a single target gene in a cell, the composition comprising a pool consisting of two, three, or four double-stranded RNA molecules each having different nucleotide sequences,
wherein the double-stranded RNA molecules of the pool are each (1) composed of two separate strands, wherein each strand is 30 nucleotides or less in length and (2) contain one strand which is complementary to a messenger RNA molecule transcribed from the target gene.

58. The composition of claim 57, wherein the individual double-stranded RNA molecules are produced by chemical synthesis.

59. The composition of claim 57, wherein members of the pool of double-stranded RNA molecules have a two nucleotide 3′ overhang.

60. The composition of claim 57, wherein individual members of the pool of double-stranded RNA molecules are 12 to 25 base pairs in length.

61. The composition of claim 57, wherein individual members of the pool of double-stranded RNA molecules are 12 to 30 base pairs in length.

62. The composition of claim 57, wherein individual members of the pool of double-stranded RNA molecules are 21-25 nucleotides in length.

63. The composition of claim 57, wherein individual members of the pool of double-stranded RNA molecules are 21-23 nucleotides in length.

64. The composition of claim 57, wherein the target gene is an endogenous gene of the cell.

65. A composition comprising a pool consisting of two, three, or four double-stranded RNA molecules each having different nucleotide sequences,
wherein all of the double-stranded RNA molecules of the pool (1) are composed of two separate strands, wherein each strand is 30 nucleotides or less in length and (2) contain one strand which is complementary to a messenger RNA molecule transcribed from a single target gene.

66. The composition of claim 65, wherein the individual double-stranded RNA molecules are produced by chemical synthesis.

67. The composition of claim 65, wherein members of the pool of double-stranded RNA molecules have a two nucleotide 3′ overhang.

68. The composition of claim 65, wherein individual members of the pool of double-stranded RNA molecules are 12 to 25 base pairs in length.

69. The composition of claim 65, wherein individual members of the pool of double-stranded RNA molecules are 12 to 30 base pairs in length.

70. The composition of claim 65, wherein individual members of the pool of double-stranded RNA molecules are 21-25 nucleotides in length.

71. The composition of claim 65, wherein individual members of the pool of double-stranded RNA molecules are 21-23 nucleotides in length.

72. The composition of claim 65, wherein the target gene is an endogenous gene of the cell.
